SEQUENCE LISTING (17 sequences)

SEQ ID NO: 1 - protein, 1368 aa, Streptococcus pyogenes [sequence omitted]

SEQ ID NO: 2 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 3 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 4 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 5 - DNA, 20 bp, Mus musculus
ggagaggacaaaaaacaaga

SEQ ID NO: 6 - DNA, 23 bp, Artificial Sequence
  mCherry
ggccacgagttcgagatcgaggg

SEQ ID NO: 7 - DNA, 117 bp, Artificial Sequence
  ssODN for mCherry
agttcatgcgcttcaaggtgcacatggagggctccgtgaattcataacttcgtatagcatacattatacgaagttatcgagggcgagggccgcccctacgagggcacccagaccgcc

SEQ ID NO: 8 - DNA, 50 bp, Artificial Sequence
  ssODN for mCherry
cgtgaacggccacgagttcgagatatcgagggcgagggcgagggccgccc

SEQ ID NO: 9 - DNA, 20 bp, Mus musculus
cagcaggtcttacccttcca

SEQ ID NO: 10 - DNA, 20 bp, Mus musculus
tacaggggttggggacataa

SEQ ID NO: 11 - DNA, 20 bp, Artificial Sequence
  Foward primer for mCherry
gagggcactaaggcagtcac

SEQ ID NO: 12 - DNA, 20 bp, Artificial Sequence
  Reverse primer for mCherry
cccatggtcttcttctgcat

SEQ ID NO: 13 - DNA, 61 bp, Mus musculus
tgaatggaaaaggagctcccaggagaggacaaaaaacaagaaggaaaaacacctctgctca

SEQ ID NO: 14 - DNA, 32 bp, Artificial Sequence
  mCherry
gtgaacggccacgagttcgagatcgagggcga

SEQ ID NO: 15 - DNA, 41 bp, Mus musculus
aggagctcccaggagaggacaaaaaacaagaaggaaaaaca

SEQ ID NO: 16 - DNA, 40 bp, Artificial Sequence
  mCherry
tccgtgaacggccacgagttcgagatcgagggcgagggcg

SEQ ID NO: 17 - DNA, 53 bp, Artificial Sequence
  ssODN for mCherry
tccgtgaattcataacttcgtatagcatacattatacgaagttatcgagggcg

(19) United States
(12) Patent Application Publication
(10) Pub. No.: US 2018/0064073 A1
(43) Pub. Date: Mar. 8, 2018

(54) Method for Transferring Cas9 mRNA Into Mammalian Fertilized Egg by Electroporation

(71) Applicants: Masakazu HASHIMOTO, Osaka (JP); Tokushima University, Tokushima (JP); BEX Co., Ltd., Tokyo (JP)

(72) Inventors: Masakazu Hashimoto, Osaka (JP); Tatsuya Takemoto, Tokushima (JP)

(73) Assignees: Tokushima University, Tokushima (JP); BEX Co., Ltd., Tokyo (JP)

(21) Appl. No.: 15/551,791

(22) PCT Filed: Feb. 18, 2016
(86) PCT No.: PCT/JP2016/054735

§ 371 Date: Aug. 17, 2017

(30) Foreign Application Priority Data
Feb. 19, 2015 (JP) 2015-031006

(51) Int. Cl. A01K 67/027; C12N 15/09; C12N 9/16

(52) U.S. Cl. CPC A01K 67/0275; C12N 9/16; C12N 15/09

(57) ABSTRACT

The disclosure relates to a method of introducing mRNA encoding Cas9 protein (Cas9 mRNA) into a mammalian embryo, comprising the steps of;
(a) placing a mixture of the mammalian embryo and a solution comprising Cas9 mRNA in the gap between a pair of electrodes, and
(b) applying a voltage to the electrodes for a voltage application duration, wherein the voltage and the voltage application duration achieve the efficiency of mRNA introduction (R) higher than the minimum required efficiency of mRNA introduction (Rₘᵢₙ) that is calculated on the basis of the concentration of Cas9 mRNA (ng/μl).

### SEQUENCE LISTING SUBMISSION VIA EFS-WEB

A computer readable text file, entitled “SequenceListing.txt,” created on or about Aug. 17, 2017, with a file size of about 48 kb contains the sequence listing for this application and is hereby incorporated by reference in its entirety.

### TECHNICAL FIELD

This application claims the benefit of priority of the prior Japanese patent application (Japanese Patent Application No. 2015-031006), the entire contents of which are incorporated herein by reference.

The disclosure relates to a method of introducing mRNA encoding Cas9 protein (Cas9 mRNA) into a mammalian embryo by electroporation. The disclosure also relates to use of the method for preparing a mammalian embryo expressing Cas9 protein, performing genome editing in a mammalian embryo, preparing a mammalian embryo whose genome is modified by genome editing, or preparing a genetically modified animal.

### BACKGROUND ART

Genetically modified animals are used for elucidating basic biological mechanisms or modeling human diseases in the fields including medical research and biology. As methods for creating genetically modified animals rapidly, processes utilizing artificial nucleases such as zinc-finger nucleases (ZFN), transcription activator-like effector nucleases (TALEN), and clustered regularly interspaced short palindromic repeat-associated system (CRISPR/Cas) have been attracted attention. These new techniques called “genome editing” have enabled to modify genomes in a wide variety of organisms without involving embryonic stem cells or induced pluripotent stem cells.

For creating a genetically modified animal by genome editing, DNA/RNA encoding an artificial nuclease has to be introduced into a pronuclear zygote. This has been achieved by microinjection, but microinjection involves disadvantage that a special skill is required for introducing DNA/RNA without disrupting the cell. Furthermore, the technique is inconvenient when numerous cells have to be treated at the same time, because DNA/RNA has to be microinjected to each pronuclear zygote one by one with a special device.

Nevertheless microinjection has been chosen in the most cases for introducing DNA/RNA into fertilized eggs. For example, linear DNAs to be inserted into genomes were microinjected into pronuclei, and circular plasmids or mRNAs for transient expression of desired genes were microinjected. Genome editing in mice, which has been established recently, is also achieved by microinjecting Cas9 mRNA and guide RNA (gRNA) or plasmids that encode the RNAs into cytoplasm or pronucleus of each embryo. The step of microinjection is rate-limiting in the generation of transgenic mice by genome editing since it requires a special skill and long time as stated above (Non-Patent Literature 1).

Electroporation is useful for introducing DNA/RNA of interest into a cell or tissue and has been applied for various organisms, for example fetal and postnatal mouse tissues including brain, testis, and muscle. However, electroporation has hardly been used for fertilized mouse eggs. It was exceptionally reported that short non-coding dsRNAs were introduced into fertilized mouse eggs by electroporation for knocking down endogenous genes (Non-Patent Literature 2), but this method is not practical because the eggs were treated with an acidic Tyrode's solution before the electroporation so that the zona pellucida was removed or thinned. The zona pellucida is essential for an embryo to be implanted and thus the treatment with the acidic Tyrode's solution is harmful. Furthermore, the method merely enabled the introduction of RNAs as short as less than 1000 bps. Another group reported that they performed electroporation without treating embryos with the acidic Tyrode's solution, but in their study only dsDNAs as short as about 500 bps were introduced into mouse embryos at the blastocyst stage (Non-Patent Literature 3).

Very recently introduction of Cas9 mRNA and gRNA into fertilized rat eggs by electroporation without the treatment of the zona pellucida has been reported (Non-Patent Literature 4). However, in the study the efficiency of the genome editing was very low as shown in the results that genomes of less than 9% of the offspring were successfully modified, despite the fact that a large amount of mRNA at the concentration of 1000 to 2000 ng/μl was used.

### REFERENCES

### Patent Literature

[Patent Literature 1] U.S. Pat. No. 8,697,359

### Non-Patent Literature

[Non-Patent Literature 1] Wang, H. et al., Cell 153, 910-918 (2013)
[Non-Patent Literature 2] Grabarek, J B. et al., Genesis 32:269-276 (2002)
[Non-Patent Literature 3] Soares, M L. et al., BMC Developmental Biology 2005, 5:28
[Non-Patent Literature 4] Kaneko, T. et al., Scientific Reports 4, 6382 (2014)
[Non-Patent Literature 5] Peng, H. et al., (2012) PLos ONE 7 (8): e43748
[Non-Patent Literature 6] Ohnishi Y. et al., Nucleic Acids Research, 2010, Vol. 38, No. 15 5141-5151
[Non-Patent Literature 7] Mazari, E. et al., Development (2014) 141, 2349-2359
[Non-Patent Literature 8] Yasue A., et al., Scientific Reports 4, 5705 (2014)
[Non-Patent Literature 9] Yang, H. et al., Cell 154, 1370-1379 (2013)
[Non-Patent Literature 10] Mashiko, D. et al., Scientific Reports 3, 3355 (2013)

### SUMMARY OF THE INVENTION

The inventors have found a suitable condition for introducing Cas9 mRNA into a mammalian embryo by electroporation.

In an aspect, provided is a method of introducing mRNA encoding Cas9 protein (Cas9 mRNA) into a mammalian embryo, comprising the steps of;

(a) placing a mixture of the mammalian embryo and a solution comprising Cas9 mRNA in the gap between a pair of electrodes, and
(b) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III)

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=882/c; Formula (A):

in which c is the concentration of Cas9 mRNA (ng/μl),

provided that the voltage is about 20 to 55 V per millimeter of the distance between the electrodes, and the product of the voltage and the voltage application duration is not more than about 990 Vmsec per millimeter of the distance between the electrodes.

In another aspect, provided is a method of preparing a mammalian embryo expressing Cas9 protein, comprising the steps of;

(a) placing a mixture of a mammalian embryo and a solution comprising Cas9 mRNA in the gap between a pair of electrodes, and
(b) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III):

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=882/c; Formula (A):

in which c is the concentration of Cas9 mRNA (ng/μl),

provided that the voltage is about 20 to 55 V per millimeter of the distance between the electrodes, and the product of the voltage and the voltage application duration is not more than about 990 Vmsec per millimeter of the distance between the electrodes.

In a further aspect, provided is a method of performing genome editing in a mammalian embryo, comprising the steps of;

(a) placing a mixture of the mammalian embryo and a solution comprising Cas9 mRNA and a further nucleic acid in the gap between a pair of electrodes, wherein the further nucleic acid is gRNA or a combination of CRISPR RNA (crRNA) and trans-activating CRISPR RNA (tracrRNA), and
(b) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III)

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=882/c; Formula (A):

in which c is the concentration of Cas9 mRNA (ng/μl),

provided that the voltage is about 20 to 55 V per millimeter of the distance between the electrodes, and the product of the voltage and the voltage application duration is not more than about 990 Vmsec per millimeter of the distance between the electrodes.

In a further aspect, provided is a method of preparing a mammalian embryo whose genome is modified by genome editing, comprising the steps of;

(a) placing a mixture of a mammalian embryo and a solution comprising Cas9 mRNA and a further nucleic acid in the gap between a pair of electrodes, wherein the further nucleic acid is gRNA or a combination of crRNA and tracrRNA, and
(b) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III)

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=882/c; Formula (A):

in which c is the concentration of Cas9 mRNA (ng/μl),

provided that the voltage is about 20 to 55 V per millimeter of the distance between the electrodes, and the product of the voltage and the voltage application duration is not more than about 990 Vmsec per millimeter of the distance between the electrodes.

In a further aspect, provided is a method of preparing a genetically modified animal, comprising the step of transferring the embryo obtained by the method mentioned above to a recipient animal.

According to the disclosure, Cas9 mRNA can be introduced into a mammalian embryo by electroporation.

### BRIEF DESCRIPTION OF DRAWINGS

FIG. 1-1 shows the amino acid sequence of SEQ ID NO: 1.

FIG. 1-2 shows the amino acid sequence of SEQ ID NO: 2.

FIG. 1-3 shows the amino acid sequence of SEQ ID NO: 3.

FIG. 1-4 shows the amino acid sequence of SEQ ID NO: 4.

FIG. 2A, FIG. 2B, FIG. 2C, FIG. 2D, and FIG. 2E illustrate the electroporation devise used in the examples.

FIG. 3A and FIG. 3B show the fluorescence intensity of mCherry in embryos electroporated under various conditions and the survival rate of the embryos at the blastocyst stage.

FIG. 4 shows the efficiency of mRNA introduction for each voltage as a function of the voltage application duration, which is expected from the results shown in FIG. 3A and FIG. 3B.

FIG. 5A, FIG. 5B and FIG. 5C show the fluorescence intensity of mCherry in embryos electroporated with pulses of the both directions and the survival rate of the embryos at the blastocyst stage.

FIG. 6A, FIG. 6B, and FIG. 6C illustrate CRISPR/Cas-mediated genome editing of Fgf10 gene, wherein the RNAs were introduced by electroporation.

FIG. 7A, FIG. 7B, and FIG. 7C show the results of genome editing wherein high concentrations of Cas9 mRNA were introduced by electroporation.

FIG. 8A, FIG. 8B, and FIG. 8C illustrate the homology directed repair (HDR) of the mCherry gene, wherein the single-stranded oligodeoxynucleotide (ssODN) was introduced by electroporation.

FIG. 9A and FIG. 9B illustrate the HDR of the mCherry gene, wherein the ssODN was introduced by electroporation.

### DETAILED DESCRIPTION

Unless otherwise defined, the terms used herein have the meaning as commonly understood to those skilled in the art in the fields including organic chemistry, medicine, pharmacology, developmental biology, cell biology, molecular biology, and microbiology. Definitions of several terms used herein are described below. The definitions herein take precedence over the general understanding.

In the disclosure when a value is accompanied with the term “about”, the value is intended to include values within range of ±10% of that value. A range defined by values of the both ends covers all values between the ends and the values of the ends. When a range is accompanied with the term “about”, it is intended that the values of the both ends are accompanied with the term “about”. For example, “about 20 to 30” means “20±10% to 30±10%”.

### Electroporation

As used herein, the term “electroporator” means a device that can generate an electric pulse. Any electroporator may be used as long as it enables steps (a) and (b) of the methods disclosed herein. Electroporators are available from manufacturers such as BioRad, BTX, BEX, Intracel, and Eppendorf.

As used herein, the term “electrode” includes any electrode that may be used for a conventional electroporation technique. For example, an electrode made of one or more metals such as platinum, gold, or aluminum may be used. Generally two electrodes are placed so that the distance between them is about 0.25 to 10 mm, for example about 0.5 to mm or about 1 to 2 mm, providing a gap between the electrodes, in which a mixture of a mammalian embryo and a solution comprising Cas9 mRNA can be placed. The two electrodes may be parts of a cuvette electrode, which also works as a container to receive the mixture. Electrodes are available from manufacturers such as BioRad, BTX, BEX, Intracel, and Eppendorf.

In the solution comprising Cas9 mRNA, the concentration of Cas9 mRNA is, for example, about 30 to 2000 ng/μl, about 50 to 1000 ng/μl, about 50 to 500 ng/μl, about 50 to 300 ng/μl, about 50 to 200 ng/μl, about 200 to about 1000 ng/μl, about 200 to 500 ng/μl, or about 200 to 300 ng/μl. In an embodiment, the solution contains Cas9 mRNA at the concentration of 200 ng/μl. Under a given electronic condition, the higher the mRNA concentration in the solution, the larger the amount of the mRNA introduced to an embryo.

Any solution that can be used for electroporation, i.e., any medium or buffer in which an embryo can survive during the electroporation, may be used for dissolving Cas9 mRNA to provide the solution used herein. For example, Opti-MEM I, PBS, HBS, HBSS, Hanks, and HCMF may be mentioned as such media or buffers. Preferably the solution contains no serum.

When the mixture of a mammalian embryo and the solution comprising Cas9 mRNA is placed in the gap between the two electrodes, the embryo and the solution may be mixed first and then added to the gap, or the embryo and the solution may be separately added to the gap. The mixture is used in the volume that the mixture can fill the gap, for example, in the volume of about 1 to 50 μl, preferably about 1.5 to 15 μl, more preferably about 2 to 10 μl. In an embodiment, the volume of the mixture is about 5 μl.

In step (b), a voltage is applied to the electrodes to achieve the efficiency of mRNA introduction (R) higher than the minimum required efficiency of mRNA introduction (Rₘᵢₙ). Rₘᵢₙ depends on the concentration of Cas9 mRNA and is calculated according to Formula (A) below:

Rₘᵢₙ=882/c; Formula (A):

in which c is the concentration of Cas9 mRNA (ng/μl).

The efficiency of mRNA introduction depends on the voltage and the voltage application duration. The efficiency of mRNA introduction is calculated according to one of the following Formulae (I) to (IV) in which t is the voltage application duration (msec):

R=0.0005×t³−0.0057×t²+0.2847×t, Formula (I):

which is employed when the voltage is about 20 to 30 V per millimeter of the distance between the electrodes;

R=0.0015×t³−0.0191×t²+0.9489×t, Formula (II):

which is employed when the voltage is about 30 to 40 V per millimeter of the distance between the electrodes;

R=0.0005×t³+0.0508×t²+0.9922×t, Formula (III)

which is employed when the voltage is about 40 to 50 V per millimeter of the distance between the electrodes;

R=0.0078×t³−0.1414×t²+3.0103×t, Formula (IV):

which is employed when the voltage is not less than 50 V per millimeter of the distance between the electrodes.

When the voltage is about 30 V per millimeter of the distance between the electrodes, Formula (II) is employed. When the voltage is about 40 V per millimeter of the distance between the electrodes, Formula (III) is employed. When the voltage is about 50 V per millimeter of the distance between the electrodes, Formula (IV) is employed.

The efficiency of mRNA introduction (R) may be any value as long as it is not less than the minimum required efficiency of mRNA introduction (Rₘᵢₙ) defined by the concentration of Cas9 mRNA. For example, when the concentration of Cas9 mRNA is 50 ng/μl, Rₘᵢₙ is 17.6, and then R may be at least 17.6, for example at least 25, preferably at least 27.1. For example, when the concentration of Cas9 mRNA is 200 ng/μl, Rₘᵢₙ is 4.41, and then R may be at least 4.41, preferably at least 7.9, more preferably at least 14.7, and most preferably at least 27.1.

For example, when the concentration of Cas9 mRNA is 50 ng/μl, Rₘᵢₙ is 17.6. In order to achieve the efficiency of mRNA introduction (R) not less than the Rₘᵢₙ value, for example, per millimeter of the distance between the electrodes, a voltage of about 20 V is applied for at least about 31 msec, a voltage of about 30 V is applied for at least about 17 msec, a voltage of about 40 V is applied for at least about 11 msec, or a voltage of about 50 V is applied for at least about 7.5 msec; preferably, a voltage of about 20 V is applied for at least about 36 msec, a voltage of about 30 V is applied for at least about 21 msec, a voltage of about 40 V is applied for at least about 14 msec, or a voltage of about 50 V is applied for at least about 11 msec; more preferably, a voltage of about 20 V is applied for at least about 37 msec, a voltage of about 30 V is applied for at least about 22 msec, a voltage of about 40 V is applied for at least about 15 msec, or a voltage of about 50 V is applied for at least about 12 msec. In an embodiment, a voltage of about 30 V per millimeter of the distance between the electrodes is applied for about 21 msec.

For example, when the concentration of Cas9 mRNA is 200 ng/μl, Rₘᵢₙ is 4.41. In order to achieve the efficiency of mRNA introduction (R) not less than the Rₘᵢₙ value, for example, per millimeter of the distance between the electrodes, a voltage of about 20 V is applied for at least about 15 msec, a voltage of about 30 V is applied for at least about 5 msec, a voltage of about 40 V is applied for at least about 3.8 msec, or a voltage of about 50 V is applied for at least about 1.6 msec; preferably, a voltage of about 20 V is applied for at least about 21 msec, a voltage of about 30 V is applied for at least about 9 msec, a voltage of about 40 V is applied for at least about 6 msec, or a voltage of about 50 V is applied for at least about 3 msec; more preferably, a voltage of about 20 V is applied for at least about 29 msec, a voltage of about 30 V is applied for at least about 15 msec, a voltage of about 40 V is applied for at least about 10 msec, or a voltage of about 50 V is applied for at least about 6 msec; most preferably, a voltage of about 20 V is applied for at least about 37 msec, a voltage of about 30 V is applied for at least about 22 msec, a voltage of about 40 V is applied for at least about 15 msec, or a voltage of about 50 V is applied for at least about 12 msec. In an embodiment, a voltage of about 30 V per millimeter of the distance between the electrodes is applied for about 21 msec.

The efficiency of mRNA introduction is increased depending on the voltage and the voltage application duration. However, when the voltage is too high or the voltage application duration is too long, survival rate of the embryo tends to decrease. The voltage per millimeter of the distance between the electrodes should be about 20 to 55 V, preferably about 20 to 40 V, more preferably about 25 to 35 V, most preferably about 30 V. The voltage application duration is determined so that the product of the voltage and the voltage application duration per millimeter of the distance between the electrodes is not more than about 990 Vmsec, preferably not more than about 810 Vmsec, more preferably not more than about 630 Vmsec.

The voltage during the electroporation may be constant or varied. In an embodiment, the voltage is constant. A conventional square pulse electroporator can be used for generating a constant voltage.

In an embodiment, the voltage is applied as multiple pulses. For example, the voltage is applied as 2 to 15, 3 to 11, 5 to 9, or 6 to 8 pulses. In an embodiment, the voltage is applied as 7 pulses. The duration of each pulse is, for example, about 0.01 to 33 msec, about 0.5 to 15 msec, about 1 to 10 msec, or about 2 to 5 msec, for example, about 3 msec. The interval between each pulse is, for example, about 0.5 to 500 msec, preferably about 5 to 250 msec, more preferably about 10 to 150 msec, still preferably about 80 to 120 msec. In an embodiment, the interval between each pulse is about 97 msec. The duration and magnitude of each pulse may be same or different.

When the voltage is applied as multiple pulses, the direction of each pulse may be same or the direction of at least one pulse may be opposite to the others. When pulses of the both directions are applied, the pulses may be applied in any order. For example, sequential pulses of one direction may be applied and followed by sequential pulses of the opposite direction, pulses of the both directions may be applied in an alternate order, or pulses of the both directions may be applied in a random order.

As used herein, the term “pulse of the opposite direction” means, compared to a pulse generated by a pair of an anode and cathode, a pulse that is generated when the anode and cathode are interchanged. Similarly, when a pair of electrodes works as an anode and cathode to generate a voltage, the term “voltage of the opposite direction” means a voltage generated by interchanging the anode and cathode.

In an embodiment, step (b) may be replaced with the following steps (c) and (d);

(c) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III)

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=441/c; Formula (B):

in which c is the concentration of Cas9 mRNA (ng/μl),

provided that the voltage is about 20 to 55 V per millimeter of the distance between the electrodes, the product of the voltage and the voltage application duration is not more than about 630 Vmsec, preferably not more than 540 Vmsec, per millimeter of the distance between the electrodes, and the voltage may be applied as two or more pulses; and

(d) applying a voltage of the opposite direction to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to one of Formulae (I) to (IV);

wherein Rₘᵢₙ is calculated according to Formula (B);

provided that the voltage is about 20 to 55 V per millimeter of the distance between the electrodes, the product of the voltage and the voltage application duration is not more than about 630 Vmsec, preferably not more than 540 Vmsec, per millimeter of the distance between the electrodes, and the voltage may be applied as two or more pulses;

wherein when the voltage is applied as two or more pulses in steps (c) and (d), the two or more pulses may be applied as sequential pulses of one direction followed by sequential pulses of the opposite direction; pulses of the both directions in an alternate order; or pulses of the both directions in a random order.

In steps (c) and (d) the voltage and the voltage application duration may be determined as described for step (b).

### Genome Editing

As used herein, “genome editing” means modifying one or more genes of a mammalian cell by using an artificial nuclease. One or both alleles are modified by the genome editing. A bacterial CRISPR/Cas system is used for the genome editing. Details of CRISPR/Cas systems are described in, for example, Wang, H. et al., Cell, 153, 910-918 (2013) and U.S. Pat. No. 8,697,359, the entire contents of which are incorporated herein by reference.

In general, genome editing with a CRISPR/Cas system requires Cas9 protein, an endonuclease, and gRNA. gRNA is a chimeric RNA in which bacterial crRNA and tracrRNA are combined. The crRNA is responsible for specificity to the target sequence and the tracrRNA works as a scaffold for Cas9 protein. When gRNA and Cas9 protein are expressed in a cell, the target sequence in the genome may be permanently modified.

The gRNA/Cas9 complex is recruited to the target sequence in the genome through complementary binding between the gRNA and the target sequence. The binding requires that a protospacer adjacent motif (PAM) is present immediately downstream of the target sequence in the genome. Cas9 protein localized to the target sequence cleaves the both strands of the genomic DNA, resulting in a double strand break (DSB). The DSB may be repaired through non-homologous end joining (NHEJ) pathway or homology directed repair (HDR) pathway. The NHEJ repair pathway frequently leads to insertion/deletion of at least a nucleotide (InDel) at the DSB site. The InDel may cause a frameshift and/or a stop codon, disrupting the open reading frame of the targeted gene. On the other hand, any desired mutation may be introduced to the target gene through the HDR pathway, because the HDR requires a DNA “repair template” and its sequence is copied to the cleaved genomic DNA.

### Cas9 Protein

Wild-type Cas9 proteins have two functional endonuclease domains, RuvC and HNH. The RuvC domain cleaves one strand of a double strand DNA and the HNH domain cleaves another strand. When the both domains are active, the Cas9 protein can generate the DSB in genomic DNA. Cas9 proteins having only one of the enzymatic activities have been developed. Such Cas9 proteins cleave only one strand of the target DNA. For example, the RuvC and HNH domains of the Cas9 protein derived from Streptococcus pyogenes are inactivated by D10A and H840A mutations, respectively.

Ability of Cas9 proteins to bind to a target DNA is independent from their ability to cleave the target DNA. Even if both of the RuvC and HNH domains are inactive and the Cas9 protein has no nuclease activity, the Cas9 protein still retains the ability to bind to the target DNA in the presence of gRNA. Accordingly, Cas9 proteins lacking nuclease activity (dCas9 proteins) may be used as a tool in molecular biology. For example, such dCas9 proteins may be used as a transcriptional regulator to activate or suppress expression of a gene through binding to a known transcriptional regulatory domain via gRNA. For example, if a dCas9 protein is fused with a transcriptional activator, it can activate transcription of the target gene. To the contrary, when only the dCas9 protein binds to the target sequence, the transcription may be suppressed. Expression of various genes may be regulated by targeting a sequence close to the promoter of the desired gene. Alternatively, in assays such as chromatin immunoprecipitation, genomic DNA may be purified by using a dCas9 protein fused with an epitope tag and a gRNA that targets any sequence in the genomic DNA. When a dCas9 protein fused with a fluorescent protein such as GFP or mcherry is used together with a gRNA that targets a desired sequence in genomic DNA, it may be used as a DNA label that can be detected in a living cell.

As used herein, the term “Cas9 protein” means a protein having an ability to bind to a DNA molecule in the presence of gRNA, including Cas9 proteins having both the RuvC and HNH nuclease activities and Cas9 proteins lacking either or both the nuclease activities. The DNA-binding activity and nuclease activity of Cas9 proteins may be measured, for example, by the method described in Samuel H. Sternberg et al., Nature 507, 62-67 (2014), the entire contents of which are incorporated herein by reference.

As used herein, the term “Cas9 mRNA” means an mRNA encoding any one of the Cas9 proteins. The Cas9 mRNA may have any nucleotide sequence as long as it is translated to an amino acid sequence of a Cas9 protein.

In an embodiment, a Cas9 protein derived from a bacterium having a CRISPR system is used. Bacteria known to have a CRISPR system include bacteria belonging to Aeropyrum sp., Pyrobaculum sp., Sulfolobus sp., Archaeoglobus sp., Halocarcula sp., Methanobacteriumn sp., Methanococcus sp., Methanosarcina sp., Methanopyrus sp., Pyrococcus sp., Picrophilus sp., Thermoplasma sp., Corynebacterium sp., Mycobacterium sp., Streptomyces sp., Aquifex sp., Porphyromonas sp., Chlorobium sp., Thermus sp., Bacillus sp., Listeria sp., Staphylococcus sp., Clostridium sp., Thermoanaerobacter sp., Mycoplasma sp., Fusobacterium sp., Azoarcus sp., Chromobacterium sp., Neisseria sp., Nitrosomonas sp., Desulfovibrio sp., Geobacter sp., Micrococcus sp., Campylobacter sp., Wolinella sp., Acinetobacter sp., Erwinia sp., Escherichia sp., Legionella sp., Methylococcus sp., Pasteurella sp., Photobacterium sp., Salmonella sp., Xanthomonas sp., Yersinia sp., Treponema sp., and Thermotoga sp. For example, a Cas9 protein derived from a bacterium such as Streptococcus pyogenes, Neisseria meningitides, Streptococcus thermophiles, or Treponema denticola is used.

In an embodiment, a Cas9 protein which is a fusion protein with at least one other protein or peptide may be used. Such proteins and peptides include, for example, fluorescent proteins, transcriptional factors, epitope tags, tags for protein purification, and nuclear localization signal peptides.

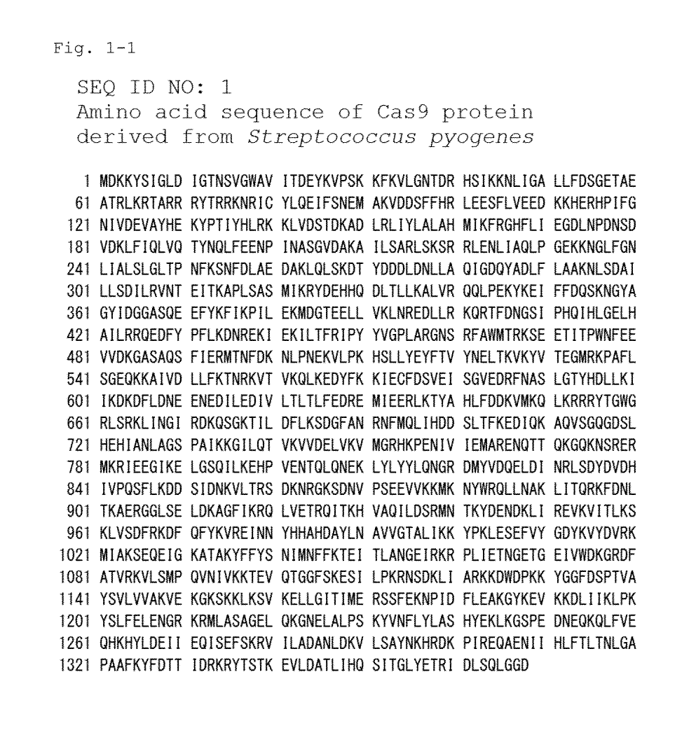

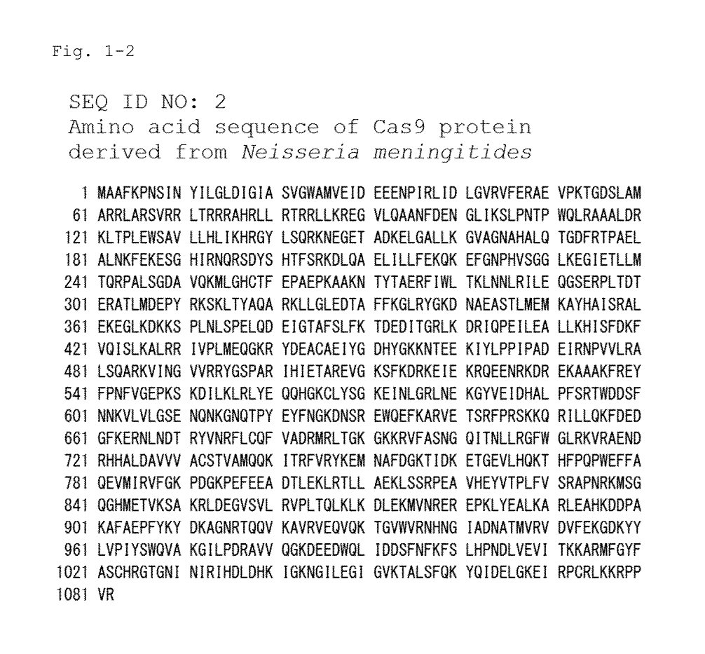

In an embodiment, a Cas9 protein may comprise an amino acid sequence having amino acid sequence identity at least about 80% with an amino acid sequence selected from SEQ ID NOs: 1 to 4 shown in FIG. 1-1, FIG. 1-2, FIG. 1-3 and FIG. 1-4 and have an ability to bind to DNA in the presence of gRNA and optionally the RuvC and/or HNH nuclease activity. For example, a Cas9 protein comprising or consisting of an amino acid sequence selected from SEQ ID NOs: 1 to 4 may be used. For example, a Cas9 protein comprising an amino acid sequence having amino acid sequence identity at least about 80% with the amino acid sequence of SEQ ID NO: 1 and having an ability to bind to DNA in the presence of gRNA and optionally the RuvC and/or HNH nuclease activity may be used. For example, a Cas9 protein comprising or consisting of the amino acid sequence of SEQ ID NO: 1 may be used. The amino acid sequences of SEQ ID NOs: 1 to 4 correspond to amino acid sequences of Cas9 proteins derived from Streptococcus pyogenes, Neisseria meningitides, Streptococcus thermophiles, and Treponema denticola, respectively.

In an embodiment, a Cas9 protein comprising an amino acid sequence having amino acid sequence identity at least about 80%, for example, at least about 85%, preferably at least about 90%, more preferably at least about 95%, still more preferably at least about 97%, still more preferably at least about 98%, still more preferably at least about 99%, still more preferably at least about 99.5% with an amino acid sequence selected from SEQ ID NOs: 1 to 4 may be used. The term “amino acid sequence identity” means the percentage of identical amino acid residues in given two amino acid sequences optimally aligned to each other. For example, 90% amino acid sequence identity means that 90% of total amino acid residues are identical between optimally aligned two amino acid sequences. Methods of aligning amino acid sequences and calculating amino acid sequence identity are known to those skilled in the art. For example, programs such as BLAST may be used.

Cas9 mRNA may be obtained by cloning a DNA coding an amino acid sequence of a desired Cas9 protein into a vector suitable for in vitro transcription and performing in vitro transcription. Vectors suitable for in vitro transcription are known to those skilled in the art. In vitro transcription vectors that contain a cloned DNA encoding a Cas9 protein are also known and include, for example, pT7-Cas9 available from Origene. Methods of in vitro transcription are known to those skilled in the art.

In an embodiment, the solution comprising Cas9 mRNA may contain at least one further nucleic acid and the nucleic acid may be introduced to an embryo together with the Cas9 mRNA. The further nucleic acid may be, for example, gRNA, crRNA, tracrRNA or ssODN. For example, gRNA alone, combination of crRNA and tracrRNA, combination of gRNA and ssODN, or combination of crRNA, tracrRNA and ssODN may be used.

The concentration ratio of gRNA to Cas9 mRNA may be 1:20 to 1:1, for example 1:2, in weight. For example, the solution may contain 200 ng/μl Cas9 mRNA and 100 ng/μl gRNA. The concentration ratio of crRNA to tracrRNA to Cas9 mRNA may be 1:1:20 to 1:1:1, for example 1:1:2, in weight. For example, the solution may contain 200 ng/μl Cas9 mRNA, 100 ng/μl crRNA and 100 ng/μl tracrRNA. The concentration of ssODN in the solution may be 200 to 1000 ng/μl, for example, 600 ng/μl.

gRNA

Genome editing requires a target-specific gRNA. As used herein, the term “guide RNA” or “gRNA” means a synthetic single-strand RNA comprising a fusion of crRNA and tracrRNA. The crRNA and tracrRNA may be linked via a linker. Cas9 protein can bind to a target sequence in genomic DNA in the presence of gRNA specific for the target sequence.

crRNA is derived from an endogenous bacterial RNA and is responsible for sequence specificity of gRNA. crRNA comprising a target sequence present in genomic DNA or the sequence compliment thereto is used herein. The target sequence is selected so that the sequence is present immediately upstream of a protospacer adjacent motif (PAM) in the genomic DNA. The target sequence may be present in either strand of the genomic DNA. Many tools are available for selecting a target sequence and/or designing gRNA, and lists of target sequences which are predicted for various genes in various species may be obtained. For example, Feng Zhang lab's Target Finder, Michael Boutros lab's Target Finder (E-CRISP), RGEN Tools: Cas-OFFinder, CasFinder: Flexible algorithm for identifying specific Cas9 targets in genomes, and CRISPR Optimal Target Finder, may be mentioned and the entire contents thereof are incorporated herein by reference.

The PAM sequence is present immediately downstream of the target sequence in the genomic DNA, but not present immediately downstream of the target sequence in the gRNA. Cas9 proteins can bind to any DNA sequence as long as the DNA has the PAM sequence immediately downstream of the target sequence. The exact sequence of the PAM is dependent upon the bacterial species from which the Cas9 protein is derived. One of the most widely used Cas9 proteins is derived from Streptococcus pyogenes and the corresponding PAM sequence is NGG present immediately downstream of the 3′ end of the target sequence. PAM sequences of various bacterial species are known, for example, Neisseria meningitides: NNNNGATT, Streptococcus thermophiles: NNAGAA, Treponema denticola: NAAAAC. In these sequences, N represents any one of A, T, G, and C.

tracrRNA hybridizes to a part of crRNA to form a hairpin loop structure. The structure is recognized by Cas9 protein and a complex of crRNA, tracrRNA and Cas9 protein is formed. Thus tracrRNA is responsible for the ability of gRNA to bind to Cas9 protein. tracrRNA is derived from an endogenous bacterial RNA and has a sequence intrinsic to the bacterial species. tracrRNA derived from the bacterial species known to have a CRISPR system listed above may be used herein. Preferably, tracrRNA and Cas9 protein derived from the same species are used. For example, tracrRNA derived from Streptococcus pyogenes, Neisseria meningitides, Streptococcus thermophiles, or Treponema denticola may be used.

gRNA may be obtained by cloning a DNA having a desired gRNA sequence into a vector suitable for in vitro transcription and performing in vitro transcription. Vectors suitable for in vitro transcription are known to those skilled in the art. In vitro transcription vectors that comprise a sequence corresponding to gRNA with no target sequence are also known in the art. gRNA may be obtained by inserting a synthesized oligonucleotide of a target sequence into such vector and performing in vitro transcription. Such vectors include, for example, pUC57-sgRNA expression vector, pCFD1-dU6:1gRNA, pCFD2-dU6:2gRNA pCFD3-dU6:3gRNA, pCFD4-U6:1_U6:3tandemgRNAs, pRB17, pMB60, DR274, SP6-sgRNA-scaffold, pT7-gRNA, DR274, and pUC57-Simple-gRNA backbone available from Addgene, and pT7-Guide-IVT available from Origene. Methods of in vitro transcription are known to those skilled in the art.

Combination of crRNA and tracrRNA may be used in place of gRNA. When the combination is used, the crRNA and tracrRNA are separate RNA molecules and the weight ratio of crRNA to tracrRNA may be 1:10 to 10:1, for example 1:1.

### Homology Directed Repair (HDR)

Combination of CRISPR/Cas system with HDR can modify one or more desired nucleotides in a target sequence. In order to utilize the HDR for gene editing, a DNA repair template containing a desired sequence is necessary. In an embodiment, the DNA repair template is a single-stranded oligodeoxynucleotide (ssODN). ssODN has homology to the sequences immediately upstream and downstream of the DSB. The length and binding position of each homology region is dependent on the size of the change to be introduced. In the presence of a suitable template, the HDR can modify the desired nucleotide at the position of the DSB made by Cas9 protein. ssODN is designed so that the modified gene is not cleaved by the Cas9 protein. This means that ssODN should not contain the PAM sequence immediately downstream of the target sequence. For example, the sequence modified by ssODN is not cleaved by Cas9 protein when the ssODN has a nucleotide sequence different from the PAM sequence at the positon corresponding to the PAM sequence. Details of methods of designing ssODNs are described in, for example, Yang, H. et al., Cell, 154(6), 1370-9 (2013), the entire contents of which are incorporated herein by reference. In general, ssODN is introduced into a cell together with gRNA and Cas9 mRNA.

As used herein, the term “introducing mRNA encoding Cas9 protein into an embryo” or “introducing Cas9 mRNA into an embryo” means introducing Cas9 mRNA to an embryo by electroporation at the amount that enables expression of Cas9 protein in the embryo or at least one cell derived from the embryo. Preferably, Cas9 mRNA is introduced at the amount that enables genome editing of at least one target gene in the genome of the embryo or at least one cell derived from the embryo in the presence of gRNA.

In an embodiment, it is confirmed that the genome editing has occurred. Whether the genome editing has occurred can be confirmed by various methods known in the art. For example, when the phenotype of the target gene is known, change of the phenotype may be detected. Alternatively, the region comprising the target sequence in the genomic DNA of the embryo or at least one cell derived from the embryo may be sequenced. In the case of HDR, a restriction enzyme site may be incorporated to ssODN and the restriction fragment length polymorphism (RFLP) may be detected. These methods are well known in the art.

As used herein, the term “mammalian” or “mammal” means any organism that is classified in the Mammalia. The mammal includes, for example, primates (e.g., monkey, human), rodents (e.g., mouse, rat, guinea pig, hamster), cattle, pig, sheep, goat, horse, dog, cat, and rabbit. In an embodiment, the mammal is a rodent. In an embodiment, the mammal is a mouse.

As used herein, the term “embryo” means an egg or embryo after a fertilization event, including a fertilized egg (one-cell stage) and early embryos from the two-cell stage to the blastocyst stage. The fertilization may occur in vivo or in vitro. Embryos may be stored frozen prior to or after the fertilization. Methods of preparing, culturing and storing embryos are known in the art. Preferably, prior to the electroporation, embryos are washed with a solution for the electroporation to remove the culture medium.

In an embodiment, the embryo is at the one-cell stage to the morula stage, preferably at the one-cell stage to the eight-cell stage, more preferably at the one-cell stage to the four-cell stage, still more preferably at the one-cell stage or the two-cell stage, for example, at the one-cell stage. In an embodiment, the electroporation is performed at least about 6 hours, preferably at least about 9 hours, more preferably at least about 12 hours after the fertilization. In an embodiment, the electroporation is performed about 6 to 18 hours, preferably about 9 to 15 hours, more preferably about 11 to 13 hours, for example about 12 hours after the fertilization. Usually, embryos have a protective membrane called zona pellucida, which can be removed or thinned e.g. by treatment with an acidic Tyrode's solution. The zona pellucida may be removed or thinned, but this is not an indispensable step herein. Preferably, the zona pellucida is not removed or thinned.

In an embodiment, the electroporated embryo is cultured and the survival of the embryo is confirmed. Methods of culturing an embryo are well known to those skilled in the art. Survival of the embryo can be confirmed by observing that at least one cell division occurred in the embryo after the electroporation.

In an embodiment, a mammalian embryo whose genome is modified by genome editing may be obtained. Another embodiment provides a method of preparing a genetically modified animal comprising the step of transferring the obtained embryo to a recipient animal. The recipient animal is usually a pseudopregnant female of the same animal species as the embryo. The embryo is usually implanted to the fallopian tube. Depending on the developmental stage of the embryo, it may be implanted to the uterus. The recipient animal implanted with the embryo delivers a genetically modified animal. Methods of preparing a genetically modified animal are known to those skilled in the art. For example, the method described in Manipulating the Mouse Embryo: A Laboratory Manual, Fourth Edition (Cold Spring Harbor Press), the entire contents of which are incorporated herein by reference, may be used.

In an embodiment, the following steps are employed;

(a) placing a mixture of a mammalian embryo and a solution comprising at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 990 Vmsec per millimeter of the distance between the electrodes.

In an embodiment, the following steps are employed;

(a) placing a mixture of a mammalian embryo and a solution comprising at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 990 Vmsec per millimeter of the distance between the electrodes.

In an embodiment, the following steps are employed;

(a) placing a mixture of a mammalian embryo and a solution comprising at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 990 Vmsec per millimeter of the distance between the electrodes.

In an embodiment, the following steps are employed;

(a) placing a mixture of a mammalian embryo and a solution comprising at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising gRNA and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising crRNA, tracrRNA and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising gRNA, ssODN and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising crRNA, tracrRNA, ssODN and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes, provided that the product of the voltage and the voltage application duration is not more than about 630 Vmsec per millimeter of the distance between the electrodes.

In an embodiment, the following steps are employed;

(a) placing a mixture of a mammalian embryo and a solution comprising gRNA and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising crRNA, tracrRNA and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising gRNA, ssODN and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising crRNA, tracrRNA, ssODN and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising gRNA and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising crRNA, tracrRNA and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising gRNA, ssODN and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo and a solution comprising crRNA, tracrRNA, ssODN and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising gRNA and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising crRNA, tracrRNA and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising gRNA, ssODN and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising crRNA, tracrRNA, ssODN and at least about 200 ng/μl Cas9 mRNA in the gap between a pair of electrodes, and
(b) applying a voltage of at least 20 V per millimeter of the distance between the electrodes for at least about 15 msec or a voltage of at least about 30 V per millimeter of the distance between the electrodes for at least about 9 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising gRNA and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising crRNA, tracrRNA and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising gRNA, ssODN and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising crRNA, tracrRNA, ssODN and at least about 200 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising gRNA and at least about 50 ng/μl Cas9 mRNA in the gap between a pair of electrodes, and (b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising crRNA, tracrRNA and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising gRNA, ssODN and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

In an embodiment, the following steps are employed;

(a) placing a mixture of a mammalian embryo at the one-cell stage and a solution comprising crRNA, tracrRNA, ssODN and at least about 50 ng/μl Cas9 mRNA in the gap between a pair of electrodes, and
(b) applying a voltage of at least about 30 V per millimeter of the distance between the electrodes for at least about 21 msec to the electrodes,

provided that the product of the voltage and the voltage application duration is not more than about 630 Vmsec per millimeter of the distance between the electrodes.

The following embodiments may be mentioned;

[1] A method of introducing mRNA encoding Cas9 protein (Cas9 mRNA) into a mammalian embryo, comprising the steps of;
(a) placing a mixture of the mammalian embryo and a solution comprising Cas9 mRNA in the gap between a pair of electrodes, and
(b) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III)

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=882/c; Formula (A):

in which c is the concentration of Cas9 mRNA (ng/μl),

provided that the voltage is about 20 to 55 V per millimeter of the distance between the electrodes, and the product of the voltage and the voltage application duration is not more than about 990 Vmsec per millimeter of the distance between the electrodes.

[2] The method according to item [1], wherein the voltage is about 20 to 40 V per millimeter of the distance between the electrodes.
[3] The method according to item [1] or [2], wherein the voltage is about 25 to 35 V per millimeter of the distance between the electrodes.
[4] The method according to any one of items [1] to [3], wherein the voltage is about 30 V per millimeter of the distance between the electrodes.
[5] The method according to any one of items [1] to [4], wherein the mRNA concentration is about 50 to 1000 ng/μl.
[6] The method according to any one of items [1] to [5], wherein the mRNA concentration is about 50 to 200 ng/μl.
[7] The method according to any one of items [1] to [6], wherein the mRNA concentration is at least about 50 ng/μl, and the efficiency of mRNA introduction is at least about 25. [8] The method according to item [7], wherein the voltage is at least about 20 V per millimeter of the distance between the electrodes, and the voltage application duration is at least about 36 msec.
[9] The method according to item [7], wherein the voltage is at least about 30 V per millimeter of the distance between the electrodes, and the voltage application duration is at least about 21 msec.
[10] The method according to item [7], wherein the voltage is at least about 40 V per millimeter of the distance between the electrodes, and the voltage application duration is at least about 14 msec.
[11] The method according to item [7], wherein the voltage is at least about 50 V per millimeter of the distance between the electrodes, and the voltage application duration is at least about 11 msec.
[12] The method according to any one of items [1] to [6], wherein the mRNA concentration is at least about 200 ng/μl, and the efficiency of mRNA introduction is at least about 4.41.
[13] The method according to item [12], wherein the voltage is at least about 20 V per millimeter of the distance between the electrodes, and the voltage application duration is at least about 15 msec.
[14] The method according to item [12], wherein the voltage is at least about 30 V per millimeter of the distance between the electrodes, and the voltage application duration is at least about 5 msec.
[15] The method according to item [12], wherein the voltage is at least about 30 V per millimeter of the distance between the electrodes, and the voltage application duration is at least about 9 msec.
[16] The method according to item [12], wherein the voltage is at least about 40 V per millimeter of the distance between the electrodes, and the voltage application duration is at least about 3.8 msec.
[17] The method according to item [12], wherein the voltage is at least about 50 V per millimeter of the distance between the electrodes, and the voltage application duration is at least about 1.6 msec.
[18] The method according to any one of items [1] to [17], wherein the voltage is constant.
[19] The method according to any one of items [1] to [18], wherein the product of the voltage and the voltage application duration is not more than about 630 Vmsec per millimeter of the distance between the electrodes.
[20] The method according to any one of items [1] to [19], wherein the voltage is applied as 2 to 15 pulses.
[21] The method according to any one of items [1] to [20], wherein the voltage is applied as 5 to 11 pulses.
[22] The method according to item [20] or [21], wherein the interval between each pulse is about 10 to 150 msec.
[23] The method according to any one of items [20] to [22], wherein the pulses are applied in one direction.
[24] The method according to any one of items [20] to [22], wherein at least one pulse is applied in a direction opposite to the others.
[25] A method of introducing Cas9 mRNA into a mammalian embryo, comprising the steps of;
(a) placing a mixture of the mammalian embryo and a solution comprising Cas9 mRNA in the gap between a pair of electrodes,
(c) applying a voltage to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to

R=0.0005×t³−0.0057×t²+0.2847×t, Formula (I):

when the voltage is about 20 to 30 V per millimeter of the distance between the electrodes;

R=0.0015×t³−0.0191×t²+0.9489×t, Formula (II):

when the voltage is about 30 to 40 V per millimeter of the distance between the electrodes;

R=0.0005×t³+0.0508×t²+0.9922×t, Formula (III)

when the voltage is about 40 to 50 V per millimeter of the distance between the electrodes; or

R=0.0078×t³−0.1414×t²+3.0103×t, Formula (IV):

when the voltage is not less than about 50 V per millimeter of the distance between the electrodes;

in which t is the voltage application duration (msec),

wherein Rₘᵢₙ is calculated according to

Rₘᵢₙ=441/c; Formula (B):

in which c is the concentration of Cas9 mRNA (ng/μl),

provided that the voltage is about 20 to 55 V per millimeter of the distance between the electrodes, the product of the voltage and the voltage application duration is not more than about 630 Vmsec per millimeter of the distance between the electrodes, and the voltage may be applied as two or more pulses; and

(d) applying a voltage of the opposite direction to the electrodes for a voltage application duration,

wherein the voltage and the voltage application duration achieve the efficiency of mRNA introduction (R) higher than the minimum required efficiency of mRNA introduction (Rₘᵢₙ),

wherein R is calculated according to one of Formulae (I) to (IV);

wherein Rₘᵢₙ is calculated according to Formula (B);

provided that the voltage is about 20 to 55 V per millimeter of the distance between the electrodes, the product of the voltage and the voltage application duration is not more than about 630 Vmsec per millimeter of the distance between the electrodes, and the voltage may be applied as two or more pulses;

wherein when the voltage is applied as two or more pulses in steps (c) and (d), the two or more pulses may be applied as sequential pulses of one direction followed by sequential pulses of the opposite direction; pulses of the both directions in an alternate order; or pulses of the both directions in a random order.

[26] The method according to any one of items [1] to [25], wherein the embryo is at the one-cell stage or the two-cell stage.
[27] The method according to any one of items [1] to [26], wherein the embryo is at the one-cell stage.
[28] The method according to any one of items [1] to [27], wherein the electroporation is performed about 12 hours after the fertilization.
[29] The method according to any one of items [1] to [28], wherein the embryo is a rodent embryo.
[30] The method according to any one of items [1] to [29], wherein the embryo is a mouse embryo.
[31] The method according to any one of items [1] to [30], wherein the Cas9 protein comprises an amino acid sequence having at least about 90% amino acid sequence identity with the amino acid sequence of any one of SEQ ID NOs: 1 to 4 and has an ability to bind to DNA in the presence of gRNA.
[32] The method according to any one of items [1] to [31], wherein the Cas9 protein has RuvC and/or HNH nuclease activity.
[33] The method according to any one of items [1] to [32], wherein the Cas9 protein comprises the amino acid sequence of any one of SEQ ID NOs: 1 to 4.
[34] The method according to any one of items [1] to [33], wherein the Cas9 protein comprises the amino acid sequence of SEQ ID NO: 1.
[35] The method according to any one of items [1] to [34], wherein the solution comprises at least one further nucleic acid and the nucleic acid is introduced to the embryo together with the Cas9 mRNA.
[36] The method according to item [35], wherein the nucleic acid is gRNA, or combination of crRNA and tracrRNA.
[37] The method according to item [35] or [36], wherein the nucleic acid is gRNA.
[38] The method according to item [36] or [37], wherein the solution further comprises ssODN.
[39] The method according to any one of items [1] to [38], further comprising culturing the electroporated embryo and confirming the survival of the embryo.
[40] A method of preparing a mammalian embryo expressing Cas9 protein, comprising introducing Cas9 mRNA into a mammalian embryo by the method according to any one of items [1] to [39].
[41] A method of performing genome editing in a mammalian embryo, comprising introducing Cas9 mRNA and a further nucleic acid into the mammalian embryo by the method according to any one of items [35] to [38].
[42] The method according to item [41], further comprising confirming whether the genome editing has occurred.
[43] A method of preparing a mammalian embryo whose genome is modified by genome editing, comprising introducing Cas9 mRNA and a further nucleic acid into a mammalian embryo by the method according to any one of items [35] to [38].
[44] A method of preparing a genetically modified animal, comprising transferring the embryo obtained by the method according to item [43] to a recipient animal.

According to the disclosure, Cas9 mRNA can be introduced into a mammalian embryo efficiently and quickly by electroporation. Electroporation advantageously results in high survival rate of the embryos and does not require any special skill and much time. For example, even a skilled technician needs at least one hour in order to introduce mRNA into each cytoplasm or pronucleus of 100 embryos by microinjection, while electroporation easily enables the same within few minutes. Furthermore, a device for electroporation is usually cheaper than that for microinjection. Thus, the disclosure is useful for improving the efficiency, speed and cost of CRISPR/Cas-mediated genome editing and thus generation of a genetically modified animal.

### EXAMPLES

### Materials and Methods

mRNA and gRNA Preparation

pCS2-mCherry was kindly provided by Dr. Noriyuki Kinoshita (NIBB, Japan). hCas9 plasmid (pX330) was purchased from Addgene (Cambridge, Mass., USA). hCas9 gene was excised from pX330, then placed downstream of SP6 promoter in pSP64 vector (Promega) (pSP64-hCas9) and used for RNA synthesis. pCS2-mCherry and pSP64-hCas9 were linearized by digestion with NotI and SalI, respectively, and used as templates for mCherry and hCas9 mRNA synthesis using an in vitro RNA transcription kit (mMESSAGE mMACHINE SP6 Transcription Kit, Ambion, Austin, Tex., USA).

A pair of oligos targeting Fgf10 or mCherry was annealed and inserted into BsaI site of pDR274 vector (Addgene). The sequences of the oligos were as follows: Fgf10 (5′-GGAGAGGACAAAAAACAAGA-3′ (SEQ ID NO: 5) and the complementary sequence) and mCherry (5′-GGCCACGAGTTCGAGATCGAGGG-3′ (SEQ ID NO: 6) and the complementary sequence). After digestion with DraI, gRNAs were synthesized using the MEGAshortscript T7 Transcription Kit (Ambion, Austin, Tex., USA).

The synthesized RNAs, mRNA and gRNAs, were purified by phenol-chloroform-isoamylalcohol extraction and isopropanol precipitation. The precipitated RNAs were dissolved in Opti-MEM I at 2-4 μg/μl, and stored at −20° C. until use. The RNAs were quantified by absorption spectroscopy and agarose gel electrophoresis. ssODNs were purchased from Sigma in dry form, dissolved in Opti-MEM I to 1 μg/μl, and stored at −20° C. until use.

### Mice

ICR (CLEA Japan, Inc.) and B6D2F1 (C57BL/6×DBA2 F1) (Japan SLC, Inc.) female mice were used. The ICR strain was mainly used for determining suitable conditions for electroporation, and the B6D2F1 strain was used for genome editing.

### Embryo Collection

Fertilized eggs were collected from the oviducts of E0.5 ICR or B6D2F1 females naturally intercrossed with males of the same strain. The figure following E corresponds to the number of days from the fertilization. E0.5 means 12 hours after the midpoint of the day of vaginal plug. The covering cumulus cells were removed by incubating in 1% hyaluronidase/M2 medium (Sigma). For the genome editing experiments targeting H2b-mCherry, the eggs were obtained from B6D2F1 females intercrossed with R26-H2b-mCherry males (RIKEN CDB, Japan). The collected eggs were pre-cultured in mWM medium (ARK Resource, Japan) or KSOM medium (95 mM NaCl, 2.5 mM KCl, 0.35 mM KH₂PO₄.7H₂O, 0.2 mM MgSO₄.7H₂O, 0.2 mM glucose, 10 mM sodium lactate, 25 mM NaHCO₃, 0.2 mM sodium pyruvate, 1.71 mM CaCl₂.2H₂O, 0.01 mM Na₂-EDTA.2H₂O, 1 mM L-glutamine, 1 mg/ml BSA) until electroporation.

### Electroporation

A pair of custom-made (BEX, Tokyo, Japan) platinum block electrodes (length: 10 mm, width: 3 mm, height: 0.5 mm, gap: 1 mm) was used (FIG. 2A). The electrodes, connected to a CUY21EDIT II (BEX) or CUY21 Vivo-SQ (BEX), were set under a stereoscopic microscope. The collected eggs cultured in mWM medium were washed with Opti-MEM I (Life technologies) three times to remove the serum-containing medium. The eggs were then placed in a line in the electrode gap filled with RNA-containing Opti-MEM I solution (total 5 μl volume), and electroporation was performed. The electroporation conditions were 30 V (3 msec pulse (ON)+97 msec interval (OFF))×7 times unless otherwise stated. After electroporation, the eggs were immediately collected from the electrode gap and subjected to four washes with M2 medium followed by two washes with mWM medium. The eggs were then cultured in mWM medium at 37° C. in a 5% CO₂ incubator until the two-cell stage.

### Fluorescent Signal Detection and Analyses

The signal intensity of the mCherry fluorescence was measured 15 hours after electroporation, using a Nipkow-disc confocal unit CSU-W1 (Yokogawa, Japan) connected to an Axio Observer Z1 inverted microscope (Zeiss, Germany). The fluorescent signal was detected by an EM-CCD camera ImageM (Hamamatsu Photonics, Japan) and the data were analyzed using the HC image software and NIH ImageJ (http://imagej.nih.gov/ij/). The signal intensity is obtained as a relative value depending on the conditions of the measurement and analysis, and can be compared only when all the conditions are same.

### Genome Editing of Fgf10 and H2b-mCherry

The Cas9 mRNA and gRNAs targeting Fgf10 or H2b-mCherry were introduced into eggs collected from B6D2F1 females by electroporation at E0.5 as described above. For the HDR-mediated knock-in study, ssODN was introduced together with the Cas9 mRNA and gRNA. The sequence of the ssODN was as follows: H2b-mCherry (5′-AGTTCATGCGCTTCAAGGTGCACATGGAGGGCTCCGTGAATTCATAACTTCGTATAGCATA CATTATACGAAGTTATCGAGGGCGAGGGCCGCCCCTACGAGGGCACCCAGACCGCC-3′ (SEQ ID NO: 7) and 5′-CGTGAACGGCCACGAGTTCGAGATATCGAGGGCGAGGGCGAGGGCCGCCC-3′ (SEQ ID NO: 8)). The surviving 2-cell-stage embryos were transferred to the oviducts of pseudopregnant females on the day of the vaginal plug. Alternatively, the embryos were cultured in vitro until the blastocyst stage (E4.5).

To investigate CRISPR/Cas9-mediated mutation in the Fgf10 or H2b-mCherry gene, the genomes were prepared from the yolk sac of the embryos. The genomic regions flanking the gRNA target were amplified by PCR using specific primers: Fgf10 Fwd (5′-CAGCAGGTCTTACCCTTCCA-3′ (SEQ ID NO: 9)) and Fgf10 Rev (5′-TACAGGGGTTGGGGACATAA-3′ (SEQ ID NO: 10)), H2b-mCherry Fwd (5′-GAGGGCACTAAGGCAGTCAC-3′ (SEQ ID NO: 11)) and H2b-mCherry Rev (5′-CCCATGGTCTTCTTCTGCAT-3′ (SEQ ID NO: 12)). The PCR amplicons of Fgf10 or H2b-mCherry were cloned into pMD20 (Takara Bio Inc., Shiga, Japan) vector. Ten plasmids from each embryo were isolated, and the genomic region was sequenced. Sequencing was performed using the BigDye terminator Cycle Sequencing Kit ver. 3.1 and ABI 3500 Genetic Analyzer (Applied Biosystems, Foster City, Calif., USA).

### Example 1

Electroporation Conditions for Introducing mRNA into a Mouse Fertilized Egg

The conditions suitable for introducing mRNA into a fertilized mouse egg without treating the zona pellucida with an acid were studied. Electroporation set-up shown in FIG. 2A was used. The platinum block electrodes (gap: 1 mm, length: 10 mm, width: 3 mm, height: 0.5 mm) (FIG. 2C), which can hold 5 μl of a solution in the gap, were set under a stereoscopic microscope (FIG. 2A, left) and connected to an electroporator (CUY21EDIT II) (FIG. 2A, right). This system can treat about 40 to 50 eggs at once. Fertilized mouse eggs were manually positioned into a line prior to electroporation. mCherry mRNA (400 ng/μl) transcribed in vitro was used for electroporation and the efficiency of the mRNA introduction was evaluated by monitoring the fluorescence intensity of mCherry and the survival rate of the embryos at the blastocyst stage. FIG. 2A shows the electroporation set-up used in this study. FIG. 2B is higher magnification of the rectangle in FIG. 2A. FIG. 2C is a schematic drawing of the platinum block electrodes, showing that the eggs were placed in the RNA solution in the gap between the electrodes. FIG. 2D is a microscopic view of the eggs set in the electrode gap. FIG. 2E is a schematic drawing of the electroporation conditions used to introduce mRNAs into fertilized mouse eggs, showing that three to eleven repeats of a square pulse of 10-50V; 3-msec pulses with 97-msec intervals were used.

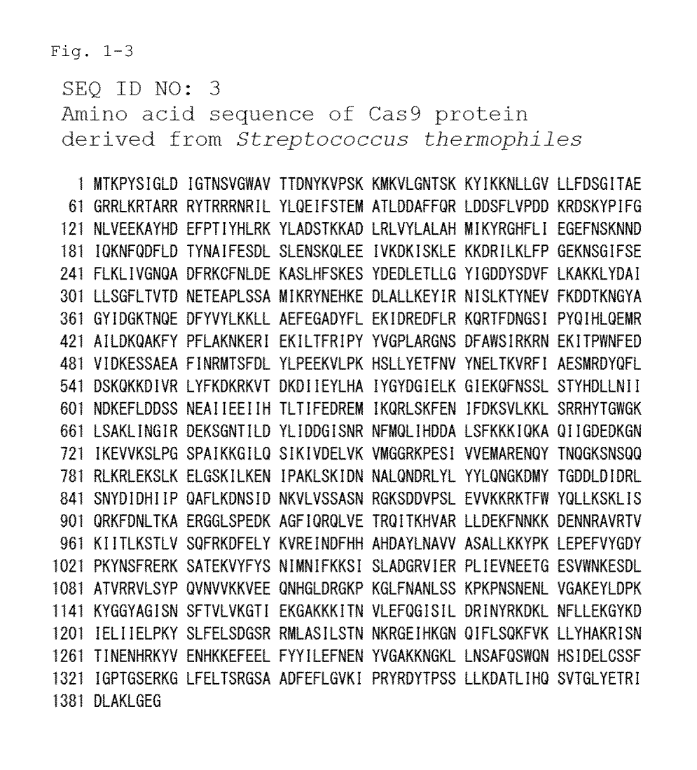

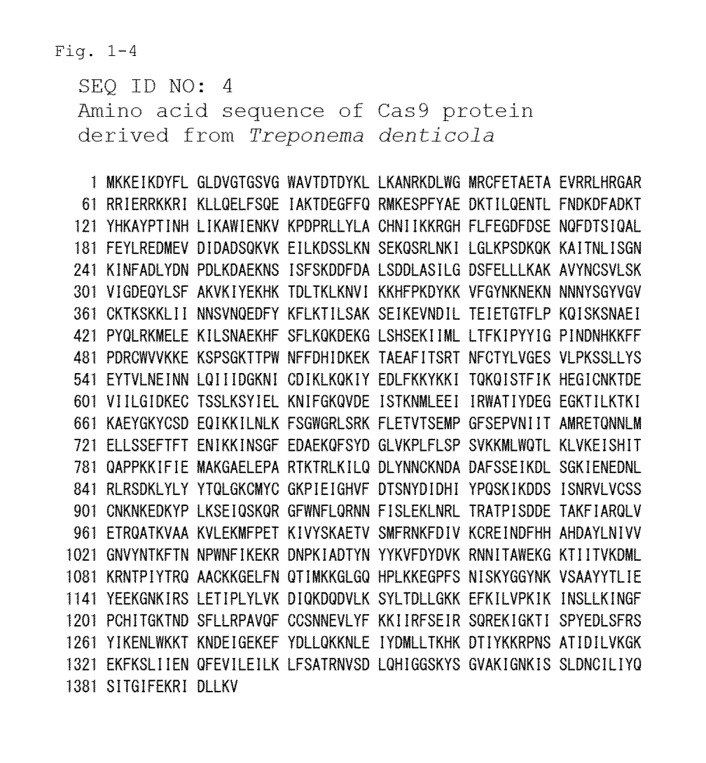

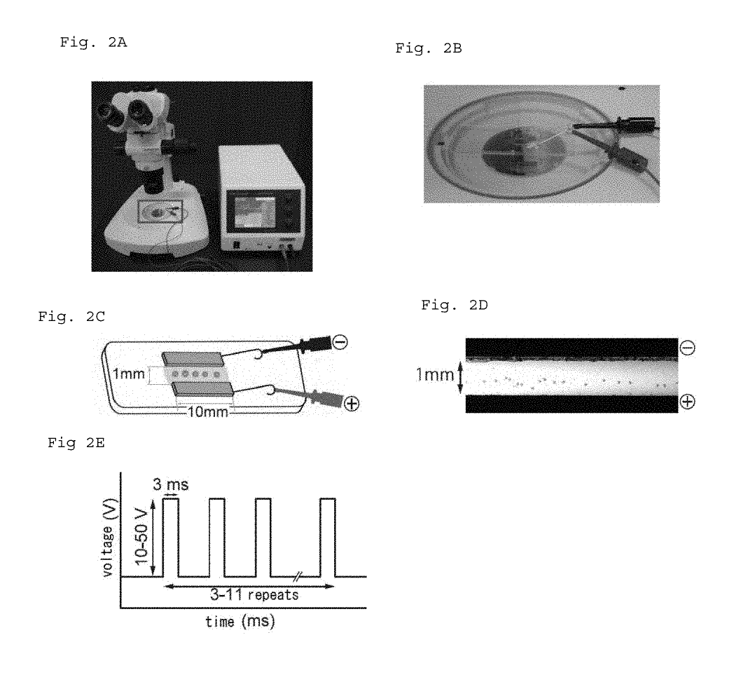

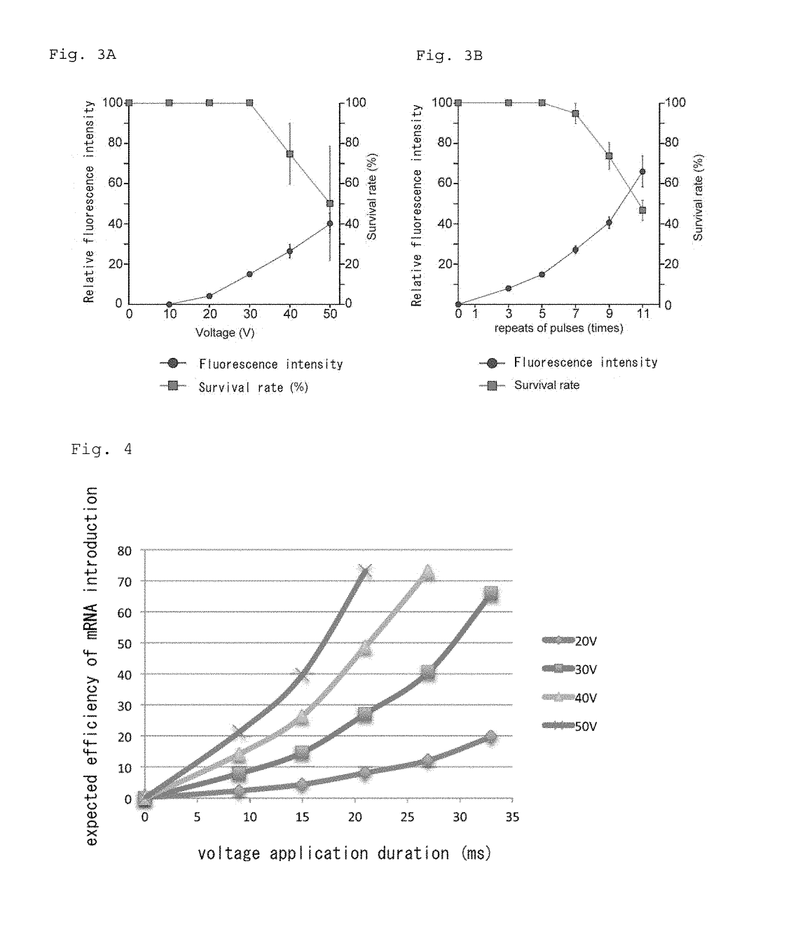

Fertilized eggs of E0.5 were electroporated at various voltages (10V-50V) while keeping the duration and number of the pulses fixed at 3 msec and five repeats, respectively. FIG. 3A shows the fluorescence intensity of mCherry (closed circles) and the survival rate of the electroporated embryos at the blastocyst stage (closed squares) plotted at the various voltages. The fluorescence was observed at the voltages of 20 V or more. The fluorescence intensity was 4.41 at the voltage of 20 V, 14.7 at the voltage of 30 V, 26.46 at the voltage of 40 V, and 39.69 at the voltage of 50 V, increasing with the voltages. Relative ratio of the fluorescence intensity at the voltage of 20 V, 30 V, 40 V, and 50 V was 0.3, 1.0, 1.8, and 2.7, respectively, when the fluorescence intensity at the voltage of 30 V was taken as 1.0. The survival rate was 100% at the voltages not more than 30 V, decreased along with the voltages at the voltage of 40 V or more, decreased to about 50% at the voltage of 50 V.

The voltage and duration of each pulse was fixed at 30 V and 3 msec, respectively, and the number of pulses was varied (x3, x5, x7, x9, and x11). FIG. 3B shows the fluorescence intensity of mCherry (closed circle) and the survival rate of the electroporated embryos at the blastocyst stage (closed squares) which were plotted as a function of the number of electroporation repeats. The fluorescence intensity increased with the number of repeats, being 7.9 at three repeats, 14.7 at five repeats, 27.1 at seven repeats, 40.6 at nine repeats, and 65.9 at eleven repeats. The survival rate of the electroporated embryos started to decrease at seven repeats and decreased to 50% at eleven repeats.

Since the fluorescence intensity of mCherry is proportional to the amount of introduced mCherry mRNA, the measured fluorescence intensity is a relative value of the efficiency of the mRNA introduction. FIG. 4 shows the expected efficiency of the mRNA introduction (R) for each voltage as a function of the voltage application duration. The expected efficiency was calculated on the basis of the fluorescence intensity shown in FIG. 3B and the relative ratio of the fluorescence intensity, which is 0.3, 1.0, 1.8, and 2.7 at the voltages of 20 V, 30 V, 40 V, and 50 V, respectively, derived from the data shown in FIG. 3A.

Mathematical functions that fit to the graph shown in FIG. 4 were constructed using Excel software. The efficiency of the mRNA introduction at each voltage may be calculated using the following functions, in which t is the voltage application duration (msec):

20 V: R=0.0005×t³−0.0057×t²+0.2847×t;

30 V: R=0.0015×t³−0.0191×t²+0.9489×t;

40 V: R=0.0005×t³+0.0508×t²+0.9922×t;

50 V: R=0.0078×t³−0.1414×t²+3.0103×t.

### Example 2

### Electroporation by Pulses of the Both Direction

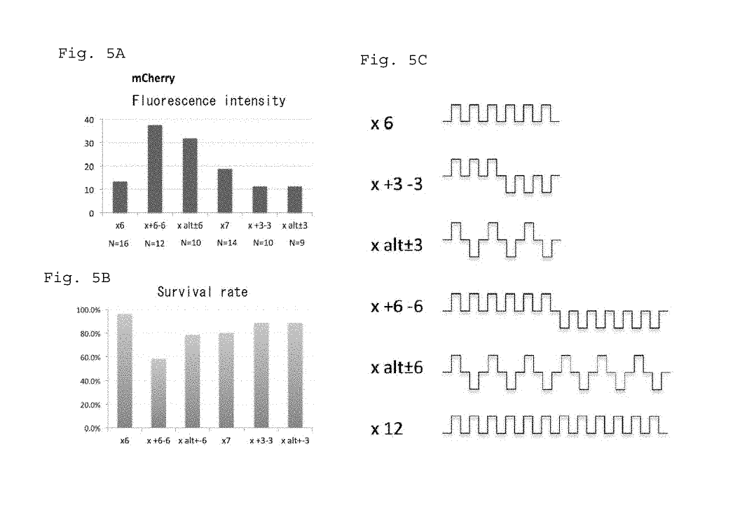

Similarly to EXAMPLE 1, mCherry mRNA was introduced to fertilized eggs of E0.5 by electroporation. The voltage and duration of each pulse were fixed at 30 V and 3 msec, respectively, and the number and direction of the pulses were changed as shown in FIG. 5C. In FIG. 5A, FIG. 5B, and FIG. 5C, “x6” indicates that six pulses of the same direction were applied, “x+3−3” indicates three pulses of one direction were sequentially applied and then three pulses of the opposite direction were applied, and “xalt±3” indicates three pulses of one direction and three pulses of the opposite direction were alternately applied. The same is applied to “x+6−6” and “xalt±6”. The fluorescence intensity of mCherry increased with the number of the pulses irrespective of direction of the voltage (FIG. 5A). The survival rate of the electroporated embryos at the blastocyst stage was high in the all cases (FIG. 5B). Especially, “x+6−6” or “xalt±6” resulted in higher survival rate than “x12”, which indicates 12 pulses of the same direction were applied.

### Example 3

Genome Editing of Fgf10 Gene by Cas9 mRNA and gRNA Introduced by Electroporation

Whether the electroporation conditions above were conducive to CRISPR/Cas9-mediated genome editing was studied.

Fgf10 gene was targeted, because Fgf10 homozygous mutant embryos have a limbless phenotype, which enables easy detection of gene destruction (Sekine, K. et al., Nature Genetics 21, 138-141(1999), the entire contents of which are incorporated herein by reference). Furthermore, it was previously confirmed that CRISPR/Cas system successfully destroyed the gene when Cas9 mRNA and gRNA were microinjected (Yasue, A. et al., Scientific Reports 4, 5705 (2014), the entire contents of which are incorporated herein by reference).

gRNA designated #563, which targets Fgf10 and comprises the nucleotide sequence of SEQ ID NO: 5, was used. The same gRNA was also used in Yasue, A. et al. Various concentrations of Cas9 mRNA and the gRNA were introduced to fertilized eggs of E0.5 by electroporation, wherein seven pulses of 30 V and 3 msec were applied. FIG. 6A shows genomic structure of the Fgf10 locus, which includes the target sequence (underlined) and the PAM sequence (AGG, capitalized), used in this study. The eggs were allowed to develop to the two-cell stage and then transferred into pseudopregnant females. The mice were dissected at E15 or E16, and phenotype of the embryos was analyzed. Depending on the limb-development defects observed at E15 or E16, the embryos were classified into three categories of phenotype: type I embryos had no limbs (Fgf10 gene knockout phenotype), type II embryos showed various defects in limb morphology (e.g., hindlimb deficiency or truncated fore- and hindlimbs), and type III embryos appeared normal. FIG. 6B shows representatives of the three categories, no limb, limb defects (left: hindlimb deficiency, right: truncated fore- and hind-limb), and normal. FIG. 6C shows a graph summarizing the effects of Cas9 and gRNA electroporation on limb development. The RNA concentrations used in each experiment are shown at left. The numbers in each row are the number of the embryos that exhibited the phenotype of each category.

Table 1 shows the concentration of the Cas9 mRNA and gRNA used, the survival rate of the embryos at the two-cell stage, and the survival rate of the embryos at E15 or E16. Table 2 shows that the Fgf10 mutant embryos were successfully generated by Cas9 mRNA and gRNA electroporation.

TABLE 1
Survival rate of electroporated embryos

| | | No. transferred | No. embryos at E15 |
|---|---|---|---|
| | | embryos/No. | or E16/No. |
| | | electroporated embryos | transferred embryos |
| Cas9 | gRNA | (survival rate at | (survival rate at |
| (ng/μl) | (ng/μl) | two-cell stage, %) | E15 or E16, %) |
| 400 | 200 | 75/80 (94) | 39/75 (52) |
| 200 | 100 | 60/63 (95) | 38/60 (63) |
| 100 | 50 | 60/64 (94) | 43/60 (72) |
| 50 | 25 | 33/35 (94) | 17/33 (51) |

TABLE 2
Defects in limb morphology in electroporated embryos

| | | | No. embryos | | |
|---|---|---|---|---|---|
| RNA conc. | | total | | Type II | |
| Cas9 | gRNA | No. | Type I | Limb | Type III |
| (ng/μl) | (ng/μl) | embryos | No limb | defect | Normal |
| 400 | 200 | 39 | 34 | 4 | 1 |
| 200 | 100 | 38 | 28 | 3 | 7 |
| 100 | 50 | 41 | 13 | 6 | 22 |
| 50 | 25 | 24 | 1 | 2 | 21 |

The survival rate of the electroporated embryos that developed to the two-cell stage (94-95%; Table 1) was much higher than embryos subjected to the microinjection method (34-35% in Yasue, A. et al.).

When 400 ng/μl Cas9 mRNA and 200 ng/μl gRNA were used for the electroporation, 97% (38/39) of the embryos displayed the characteristic limb defects. Among them 34 embryos completely lacked both fore- and hindlimbs, as expected from the Fgf10 homozygous mutant phenotype obtained by conventional gene targeting. Four embryos displayed various other limb defects. When 200 ng/μl Cas9 mRNA and 100 ng/μl gRNA were used for the electroporation, 82% (31/38) of the embryos displayed limb defects. When 100 ng/μl Cas9 mRNA and 50 ng/μl gRNA were used, 46% (19/41) of the embryos displayed at least partial limb defects. When 50 ng/μl Cas9 mRNA and 25 ng/μl gRNA were used, most of the embryos appeared normal.

To reveal whether the Fgf10 gene was disrupted in the embryos, the genomic sequence of the embryo was analyzed. The genomic region flanking the target sequence was amplified and sequenced for ten clones each from four randomly selected embryos. The wild-type sequence of the genomic region flanking the target sequence is tgaatggaaaaggagctcccaggagaggacaaaaaacaagaAGGaaaaacacctctgctca (the target sequence is underlined. Capital letters indicate PAM sequence (AGG)) (SEQ ID NO: 13). The results are shown in Table 3.

TABLE 3
Sequence analysis of Fgf10 mutants

| RNA conc. | | | |
|---|---|---|---|
| (ng/μl) | Embryo | | No. |
| Cas9/gRNA | ID | Type of mutation | clones |
| 400/200 | #1 | 15 bp deletion | 5 |
| | | 26 bp deletion | 3 |
| | | 3 bp deletion | 2 |
| | #2 | 13 bp deletion | 6 |
| | | 14 bp deletion | 4 |
| | #3 | 38 bp deletion | 4 |
| | | 6 bp deletion | 4 |
| | | 14 bp deletion | 1 |
| | | 1 bp insertion | 1 |
| | #4 | 10 bp deletion | 2 |
| | | 15 bp deletion | 2 |
| | | 14 bp deletion | 2 |
| | | 1 bp insertion | 2 |
| 200/100 | #12 | 13 bp deletion | 3 |
| | | 10 bp deletion | 3 |
| | | 13 bp deletion | 2 |
| | | 3 bp insertion | 1 |
| | | 1 bp insertion | 1 |
| | #13 | 7 bp deletion | 2 |
| | | 1 bp insertion | 2 |
| | | 1 bp insertion | 2 |
| | | 15 bp deletion | 1 |
| | | 1 bp deletion | 1 |
| | #14 | 15 bp deletion | 5 |
| | | 6 bp deletion | 2 |
| | | 15 bp deletion | 1 |
| | #15 | 1 bp insertion | 5 |
| | | 13 bp deletion | 3 |
| | | 47 bp or more deletion | 1 |
| 100/50 | #16 | 13 bp deletion | 5 |
| | | 1 bp insertion | 5 |
| | #17 | 13 bp deletion | 8 |
| | | 3 bp deletion | 1 |
| | #18 | wild type | 8 |
| | | 15 bp deletion | 2 |
| | #19 | wild type | 8 |
| | | 1 bp mutation | 1 |
| 50/25 | #28 | wild type | 8 |
| | | 2 bp insertion | 1 |
| | | 1 bp mutation | 1 |
| | #29 | wild type | 7 |
| | | 3 bp deletion | 3 |
| | #30 | wild type | 5 |
| | | 1 bp mutation | 2 |
| | | 2 bp insertion | 2 |
| | | 2 bp deletion | 1 |
| | #31 | wild type | 8 |
| | | 1 bp mutation | 1 |
| | | 1 bp mutation | 1 |

In the table, when the same type of mutation is listed twice or more for one embryo, the sequences of each clone are different.

When 400 ng/μl Cas9 mRNA and 200 ng/μl gRNA were used, all of the sequenced clones carried nucleotide insertion or deletion (indel) or mutation, and no wild-type sequence was detected. These results indicate that both alleles of the Fgf10 gene were disrupted when 400 ng/μl Cas9 mRNA and 200 ng/μl gRNA were used for the electroporation. Furthermore, each embryo had not more than four types of mutation, suggesting that the genome editing occurred immediately after the electroporation at the one-cell or two-cell stage. When 50 ng/μl Cas9 mRNA and 25 ng/μl gRNA were used, the most of the embryos appeared normal, but sequencing revealed that some clones derived from the embryos carried an indel or mutation.

The results indicate that electroporation can be used for CRISPR/Cas-mediated genome editing and the efficiency of the genome editing depends on the RNA concentration. The high concentration of Cas9 mRNA and gRNA disrupted both alleles in the almost all cells, whereas the low concentration gave chimeric embryos comprising mutant cells, in which either or both of the alleles are mutated, and wild-type cells.

### Example 4

Genome Editing of mCherry Gene by Cas9 mRNA and gRNA Introduced by Electroporation

Fertilized eggs carrying a Histone H2b (H2b)-mCherry gene inserted into the ROSA26 locus were used (Abe et al., Genesis 49, 579-590 (2011), the entire contents of which are incorporated herein by reference). The embryos developed from the eggs ubiquitously express H2b-mCherry under the control of the Rosa26 promoter, exhibiting the mCherry fluorescence in the nucleus of the all cells at the four to eight-cell stages. When genome editing targeting H2b-mCherry occurs and the gene is disrupted, the mCherry fluorescence disappears.

In this study the lowest RNA concentration required for genome editing was determined when the voltage, duration and number of pulses of electroporation was fixed at 30 V, 3 msec and seven repeats, respectively. Cas9 mRNA and gRNA targeting H2b-mCherry were introduced to the fertilized eggs of E0.5. The embryos were cultured in KSOM medium to the blastocyst stage (E4.5) and the mCherry fluorescence in the nuclei was detected. When 200 ng/μl or more of Cas9 mRNA and 100 ng/μl or more of mCherry gRNA were used, no mCherry fluorescence was detected. When 50 to 100 ng/μl Cas9 mRNA and 25 to 50 ng/μl gRNA were used, some blastomeres were mCherry-negative and others were positive. Electroporation using 25 ng/μl Cas9 mRNA and 12.5 ng/μl gRNA had no effect on the H2b-mCherry expression, suggesting that the concentrations were too low to cause the genome editing under the fixed electroporation conditions employed herein.

Further experiments were for determining which concentration of Cas9 mRNA and gRNA was important for the success of genome editing. When the concentration of Cas9 mRNA was fixed at 25 ng/μl and the concentration of gRNA was varied, genome editing did not occur even if gRNA concentration as high as 200 ng/μl was used. On the other hand, when 200 ng/μl Cas9 mRNA was used, genome editing did occur even if gRNA was decreased to 10 ng/μl. The same result was obtained when gRNA targeting the Fgf10 gene was used. The results suggest that the success of genome editing depends not on the concentration of gRNA, but on the concentration of Cas9 mRNA.

### Example 5

Genome Editing Mediated by High Concentration of Cas9 mRNA Introduced by Electroporation

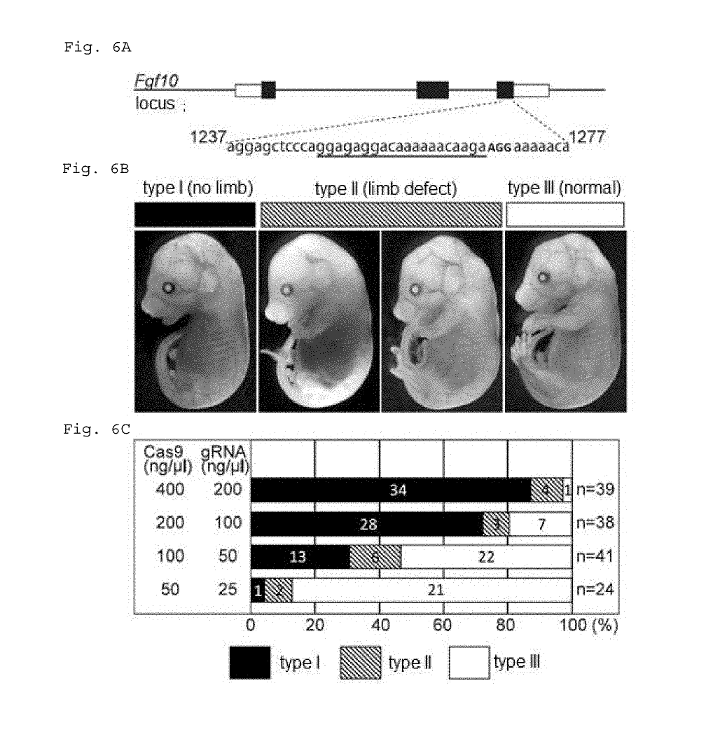

Similarly to EXAMPLE 4, electric conditions required for achieving genome editing when 2000 ng/μl Cas9 mRNA and 1000 ng/μl gRNA were used was determined. Cas9 mRNA and gRNA was introduced to fertilized mouse eggs of E0.5. The embryos were cultured in vitro to the blastocyst stage (E4.5) and the mCherry fluorescence in the nuclei was detected. The results are shown in FIG. 7A, FIG. 7B, FIG. 7C. The mCherry fluorescence was detected in all blastomeres when electroporation was not performed (FIG. 7A). When electroporation was performed by applying pulses of 30 V, 0.05 msec and two repeats, the mCherry fluorescence was not detected in some blastomeres (FIG. 7B). When electroporation was performed by applying pulses of 30 V, 0.10 msec and two repeats, the fluorescence was not detected in any blastomere (FIG. 7B). When electroporation was performed by applying pulses of 20 V, 0.05 msec and two repeats, the fluorescence was detected in all blastomeres (FIG. 7C). When electroporation was performed by applying pulses of 20 V, 0.20 msec and two repeats, the fluorescence was not detected in some blastomeres (FIG. 7C, arrow heads). When electroporation was performed by applying pulses of 20 V, 1.00 msec and two repeats, the fluorescence was not detected in any blastomere (FIG. 7C).

### Example 6

### Electroporation Condition for Achieving Genome Editing

Similarly to EXAMPLE 4, electric conditions required for achieving genome editing when 200 ng/μl Cas9 mRNA and 100 ng/μl gRNA were used was determined. Cas9 mRNA and gRNA was introduced to 5 to 12 fertilized eggs of E0.5 at the same time by electroporation using various voltages (20 to 50 V) and durations of pulses (6 to 33 msec). When electroporation conditions shown in Table 4 were employed, the mCherry fluorescence was not detected in some blastomeres of the embryos of E4.5, suggesting that genome editing was achieved.

TABLE 4
Electroporation conditions under
which genome editing was achieved

| Voltage (V) | Duration (msec) |
|---|---|
| 20 | 15 |
| 20 | 18 |
| 20 | 30 |
| 20 | 33 |
| 30 | 9 |
| 30 | 12 |
| 30 | 15 |
| 30 | 21 |
| 30 | 24 |
| 50 | 6 |
| 50 | 9 |

When 200 ng/μl Cas9 mRNA was used, genome editing was achieved by applying voltage of 20 V for 15 msec. As measured in EXAMPLE 1, the efficiency of mRNA introduction under this electric condition is 4.41.

### Example 7

Introduction of ssODN by Electroporation to Lead Homology Directed Repair

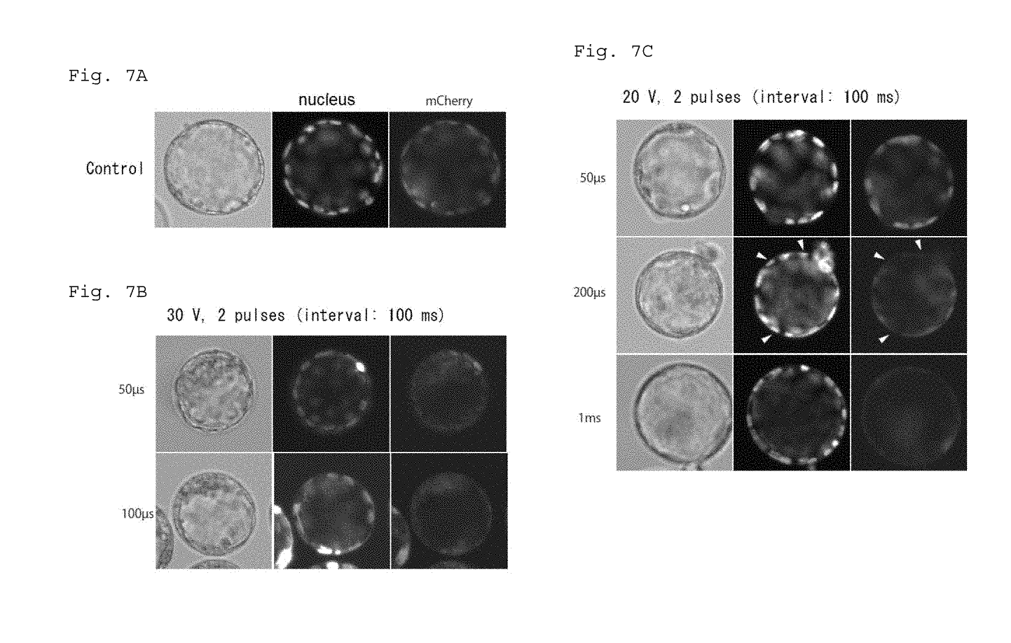

Whether electroporation could deliver ssODNs to a fertilized mouse egg and generate HDR-mediated knock-in alleles was examined. ssODN of 117 bases harboring loxP and EcoRI recognition sequences (37 bases) flanked by 40-base homologous arms was used (SEQ ID NO: 7). Cas9 mRNA, gRNA targeting the mCherry gene and the ssODN were introduced into the fertilized eggs that carry a Histone H2b (H2b)-mCherry gene inserted into the ROSA26 locus (see EXAMPLE 4) by electroporation. The embryos were cultured to the two-cell stage and transferred to pseudopregnant females. FIG. 8A shows a schematic drawing of the target sequence and the ssODN designed to insert the 37-base loxP sequence and EcoRI recognition site. The allele replaced by the ssODN would be functionally null due to the introduction of a stop codon in the loxP sequence, causing the disappearance of the nuclear mCherry fluorescence. Replacement by the ssODN was screened by Restriction Fragment Length Polymorphism (RFLP) analysis after EcoRI digestion.

All of the electroporated embryos (11/11) exhibited a loss of mCherry fluorescence. FIG. 8B shows representative images of the embryo subjected to the electroporation. The mCherry fluorescence completely disappeared in the electroporated embryo, while the control embryo, which was not subjected to the electroporation, displayed the fluorescent signals. FIG. 8C shows the results of the RFLP analysis of the collected embryos. The EcoRI-inserted alleles were digested into two bands (138 bps and 374 bps). The intact allele had 497 bps. The digested bands were observed in embryos #3, #6, #8, and #9, indicating that the four embryos were positive for EcoRI digestion among the embryos that lost the fluorescent. The unexpected bands in #1 and #7 indicate that a large deletion was generated in the target gene.

Table 5 shows the results of the sequence analysis of the embryos positive for EcoRI digestion. The wild-type sequence of the genomic regions flanking the target sequence is gtgaacggccacgagttcgagatcgaGGGcga (the target sequence is underlined. Capital letters indicate PAM sequence (GGG)) (SEQ ID NO: 14).

TABLE 5
Sequence analysis of embryos subjected to HDR-
mediated knock-in of loxP and EcoRI site

| Embryo | | No. |
|---|---|---|
| ID | Type of mutation | clones |
| #3 | HDR-mediated knock-in | 10/10 |
| #6 | HDR-mediated knock-in | 3/9 |
| | 1 bp insertion | 3/9 |
| | unexpected insertion of ssODN | 2/9 |
| | 1 bp deletion | 1/9 |
| #8 | HDR-mediated knock-in | 5/8 |
| | 1 bp deletion | 2/8 |
| | unexpected insertion of ssODN | 1/8 |
| #9 | HDR-mediated knock-in | 6/8 |
| | 1 bp deletion | 2/8 |

Sequencing revealed that all the four embryos carried the HDR-mediated replaced allele. Among them, three embryos (#6, #8, and #9) carried one to three types of alleles with indels, in addition to the replaced allele. The remaining embryo (#3) carried only the replaced allele, indicating that all of the cells carried an allele with the HDR-mediated replacement sequence.

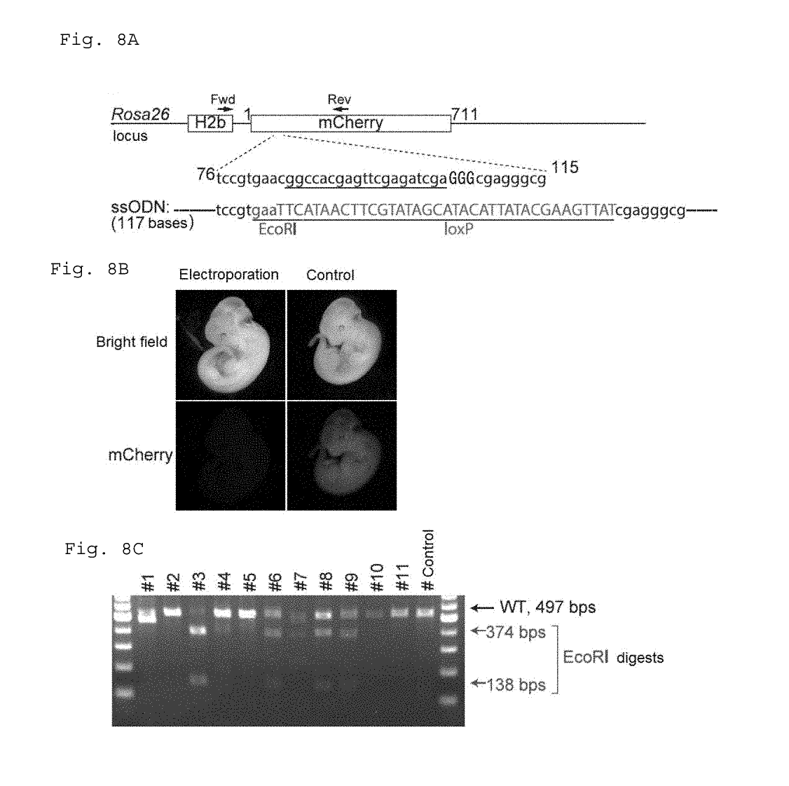

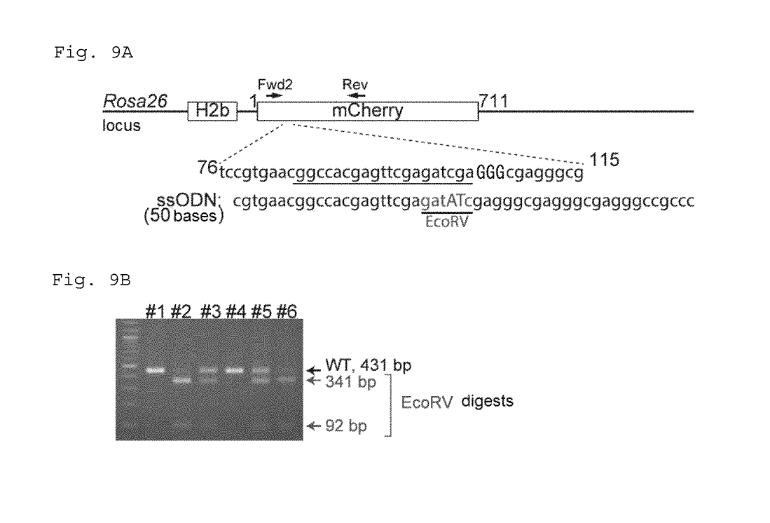

In further experiments, HDR-mediated knock-in of an EcoRV site into the mCherry gene was also achieved (Table 6 and FIG. 9A and FIG. 9B). FIG. 9A shows a schematic drawing of the target sequence and the ssODN designed to insert the EcoRV recognition site (SEQ ID NO: 8). FIG. 9B shows the results of the RFLP analysis of the collected embryos. The EcoRV-inserted alleles were digested into two bands (341 bps and 92 bps). The intact allele had 431 bps. The digested bands were observed in embryos #2, #3, #5, and #6.

Table 6 shows the results of the sequence analysis of the embryos. The wild-type sequence of the genomic regions flanking the target sequence is gtgaacggccacgagttcgagatcgaGGGcga (the target sequence is underlined. Capital letters indicate PAM sequence (GGG)) (SEQ ID NO: 14).

TABLE 6
Sequence analysis of embryos subjected
to HDR-mediated knock-in of EcoRV site

| Embryo | | No. |
|---|---|---|
| ID | Type of mutation | clones |
| #1 | 1 bp insertion | 4/10 |
| | 1 bp insertion | 3/10 |
| | 1 bp insertion | 3/10 |
| #2 | HDR-mediated knock-in | 10/10 |
| #3 | 1 bp deletion | 5/9 |
| | HDR-mediated knock-in | 3/9 |
| | 4 bp deletion | 1/9 |
| #4 | 1 bp deletion | 5/10 |
| | 1 bp insertion | 1/10 |
| | unexpected insertion of ssODN | 3/10 |
| #5 | 4 bp deletion | 3/10 |
| | HDR-mediated knock-in | 6/10 |
| | wild type | 1/10 |
| #6 | HDR-mediated knock-in | 1/9 |
| | unexpected insertion of ssODN | 1/9 |
| | unexpected insertion of ssODN | 7/9 |

In the table, when the same type of mutation is listed twice or more for one embryo, the sequences of each clone are different.

In further experiments, the HDR-mediated knock-in of an XbaI site into the Fgf10 gene was also achieved (data not shown). The results indicate that not only Cas9 mRNA and gRNA but also ssODN can be introduced to embryos by electroporation and HDR-mediated knock-in alleles are generated.

1. A method of introducing mRNA encoding Cas9 protein (Cas9 mRNA) into a mammalian embryo, comprising the steps of:
(a) placing a solution comprising the mammalian embryo and Cas9 mRNA in the gap between a pair of electrodes, and
(b) applying a voltage to the electrodes for a voltage application duration, wherein the voltage and the voltage application duration achieve the efficiency of mRNA introduction (R) higher than the minimum required efficiency of mRNA introduction (Rₘᵢₙ), wherein R is calculated according to
R=0.0005×t³−0.0057×t²+0.2847×t, Formula (I):
when the voltage is about 20 to 30 V per millimeter of the distance between the electrodes;
R=0.0015×t³−0.0191×t²+0.9489×t, Formula (II):
when the voltage is about 30 to 40 V per millimeter of the distance between the electrodes;
R=0.0005×t³+0.0508×t²+0.9922×t, Formula (III)
when the voltage is about 40 to 50 V per millimeter of the distance between the electrodes; or
R=0.0078×t³−0.1414×t²+3.0103×t, Formula (IV):
when the voltage is not less than about 50 V per millimeter of the distance between the electrodes;
in which t is the voltage application duration (msec),
wherein Rₘᵢₙ is calculated according to
Rₘᵢₙ=882/c; Formula (A):
in which c is the concentration of Cas9 mRNA (ng/μl),
provided that the voltage is about 20 to 55 V per millimeter of the distance between the electrodes, and the product of the voltage and the voltage application duration is not more than about 990 Vmsec per millimeter of the distance between the electrodes.

2. The method according to claim 1, wherein the voltage is about 25 to 35 V per millimeter of the distance between the electrodes.

3. The method according to claim 1, wherein the voltage is about 30 V per millimeter of the distance between the electrodes.

4. The method according to claim 1, wherein the concentration of Cas9 mRNA is at least about 50 ng/μl, the voltage is at least about 20 V per millimeter of the distance between the electrodes, and the voltage application duration is at least about 36 msec.

5. The method according to claim 1, wherein the concentration of Cas9 mRNA is at least about 50 ng/μl, the voltage is at least about 30 V per millimeter of the distance between the electrodes, and the voltage application duration is at least about 21 msec.

6. The method according to claim 1, wherein the concentration of Cas9 mRNA is at least about 200 ng/μl, the voltage is at least about 20 V per millimeter of the distance between the electrodes, and the voltage application duration is at least about 15 msec.

7. The method according to claim 1, wherein the concentration of Cas9 mRNA is at least about 200 ng/μl, the voltage is at least about 30 V per millimeter of the distance between the electrodes, and the voltage application duration is at least about 5 msec.

8. A method of introducing Cas9 mRNA into a mammalian embryo, comprising the steps of:
(a) placing a solution comprising the mammalian embryo and Cas9 mRNA in the gap between a pair of electrodes, and
(c) applying a voltage to the electrodes for a voltage application duration,
wherein the voltage and the voltage application duration achieve the efficiency of mRNA introduction (R) higher than the minimum required efficiency of mRNA introduction (Rₘᵢₙ),
wherein R is calculated according to
R=0.0005×t³−0.0057×t²+0.2847×t, Formula (I):
when the voltage is about 20 to 30 V per millimeter of the distance between the electrodes;
R=0.0015×t³−0.0191×t²+0.9489×t, Formula (II):
when the voltage is about 30 to 40 V per millimeter of the distance between the electrodes;
R=0.0005×t³+0.0508×t²+0.9922×t, Formula (III)
when the voltage is about 40 to 50 V per millimeter of the distance between the electrodes; or
R=0.0078×t³−0.1414×t²+3.0103×t, Formula (IV):
when the voltage is not less than about 50 V per millimeter of the distance between the electrodes;
in which t is the voltage application duration (msec),
wherein Rₘᵢₙ is calculated according to
Rₘᵢₙ=441/c; Formula (B):
in which c is the concentration of Cas9 mRNA (ng/μl),
provided that the voltage is about 20 to 55 V per millimeter of the distance between the electrodes, the product of the voltage and the voltage application duration is not more than about 630 Vmsec per millimeter of the distance between the electrodes, and the voltage may be applied as two or more pulses; and
(d) applying a voltage of the opposite direction to the electrodes for a voltage application duration,
wherein the voltage and the voltage application duration achieve the efficiency of mRNA introduction (R) higher than the minimum required efficiency of mRNA introduction (Rₘᵢₙ),
wherein R is calculated according to one of Formulae (I) to (IV);
wherein Rₘᵢₙ is calculated according to Formula (B);
provided that the voltage is about 20 to 55 V per millimeter of the distance between the electrodes, the product of the voltage and the voltage application duration is not more than about 630 Vmsec per millimeter of the distance between the electrodes, and the voltage may be applied as two or more pulses;
wherein when the voltage is applied as two or more pulses in steps (c) and (d), the two or more pulses may be applied as sequential pulses of one direction followed by sequential pulses of the opposite direction; pulses of the both directions in an alternate order; or pulses of the both directions in a random order.

9. The method according to claim 1, wherein the embryo is a rodent embryo.

10. The method according to claim 1, wherein the embryo is a mouse embryo.

11. The method according to claim 1, wherein the Cas9 protein comprises an amino acid sequence having at least about 90% amino acid sequence identity with the amino acid sequence of any one of SEQ ID NOs: 1 to 4 and has an ability to bind to DNA in the presence of gRNA.

12. The method according to claim 1, wherein the Cas9 protein has RuvC and/or HNH nuclease activity.

13. The method according to claim 1, wherein the Cas9 protein comprises the amino acid sequence of any one of SEQ ID NOs: 1 to 4.

14. The method according to claim 1, wherein the Cas9 protein comprises the amino acid sequence of SEQ ID NO: 1.

15. The method according to claim 1, wherein the solution comprises at least one further nucleic acid, the nucleic acid is gRNA or combination of crRNA and tracrRNA, and the nucleic acid is introduced to the embryo together with the Cas9 mRNA.

16. The method according to claim 15, wherein the further nucleic acid is gRNA.

17. The method according to claim 15, wherein the solution further comprises single-stranded oligodeoxynucleotide (ssODN).

18. A method of preparing a mammalian embryo expressing Cas9 protein, comprising introducing Cas9 mRNA into a mammalian embryo by the method according to claim 1.

19. A method of performing genome editing in a mammalian embryo, comprising introducing Cas9 mRNA and a further nucleic acid into the mammalian embryo by the method according to claim 15.

20. A method of preparing a mammalian embryo whose genome is modified by genome editing, comprising introducing Cas9 mRNA and a further nucleic acid into a mammalian embryo by the method according to claim 15.

21. A method of preparing a genetically modified animal, comprising transferring the embryo obtained by the method according to claim 20 to a recipient animal.
